(19) United States
(12) Patent Application Publication
(10) Pub. No.: US 2015/0328109 A1
(43) Pub. Date: Nov. 19, 2015

(54) DERMAL COMPOSITIONS CONTAINING UNNATURAL HYGROSCOPIC AMINO ACIDS

(71) Applicants: MEDPHARM LIMITED, Guildford (GB); UNIVERSITY OF READING, Reading (GB)

(72) Inventors: Natasha Arezki, Reading (GB); Andre Cobb, Reading (GB); Adrian Christopher Williams, Reading (GB); Marc Barry Brown, Guildford (GB)

(21) Appl. No.: 14/442,076

(22) PCT Filed: Nov. 12, 2013
(86) PCT No.: PCT/GB2013/052973

§ 371 Date: May 11, 2015

(30) Foreign Application Priority Data
Nov. 12, 2012 (GB) 1220354.3

(51) Int. Cl. A61K 8/44; A61K 8/49; A61K 31/198; A61K 45/06; A61Q 19/00; A61K 47/18

(52) U.S. Cl. CPC A61K 8/44; A61Q 19/007; A61K 47/183; A61K 31/198; A61K 45/06; A61K 8/4913; A61K 2800/5922

(57) ABSTRACT

Unnatural, hygroscopic amino acids are useful to enhance the moisture retention and uptake properties of skin. In particular, such amino acids are N-hydroxyserine, N-hydroxyglycine, L-homoserine, alpha-hydroxyglycine, 2-(aminooxy)-2-hydroxyacetic acid, 2-hydroxy-2-(hydroxyamino) acetic acid, 2-(aininooxy)acetic acid, and combinations thereof.

The present application relates to substances and compositions suitable to enhance hydration and moisturisation of the skin.

Xerosis, or dry skin, is a common condition experienced by most people at some point in their life. Seasonal xerosis is common during the cold, dry winter months, and evidence shows that xerosis becomes more prevalent with age (Whit-Chu, 2011). Many inflammatory skin conditions, such as atopic dermatitis, irritant contact dermatitis, and psoriasis, cause localised areas of xerotic skin. In addition, some patients have hereditary disorders, such as ichthyosis, resulting in chronic dry skin.

An important role of natural moisturising factor (NMF) is to maintain adequate skin hydration. Adequate hydration of the stratum corneum serves three major functions: (1) it maintains plasticity of the skin, protecting it from damage; (2) it allows hydrolytic enzymes to function in the process of desquamation (Rawlings, 1994), and (3) it contributes to optimum stratum corneum barrier function.

NMF is composed principally of free amino acids, and various derivatives of these amino acids such as sodium pyrrolidone carboxylic acid (pyroglutamate, 2-oxo-pyrrolidone carboxylic acid, or PCA), urocanic acid (a natural absorber of ultraviolet [UV] light), inorganic salts, sugars, and lactic acid and urea (Table 2) (Clar, 1981). Inorganic salts so far associated with NMF include the chlorides, phosphates, and citrates of sodium, potassium, calcium, and magnesium. NMF is packaged within the corneocytes, making up approximately 10 percent of the corneocyte mass and 20 percent to 30 percent of the dry weight of the stratum corneum.

NMF components are highly efficient humectants that attract and bind water from the atmosphere, or from deeper skin layers, drawing it into the corneocytes. This process can occur even at a relative humidity as low as 50 percent, allowing the corneocytes to maintain an adequate level of water in low-humidity environments. The water absorption is so efficient that NMF essentially dissolves within the water that it has absorbed (Rawlings, 1994). Hydrated NMF, particularly the neutral and basic amino acids, forms ionic interactions with keratin fibres, reducing the intermolecular forces between the fibres and, thus, increasing the elasticity of the stratum corneum. This elasticity serves to make the skin appear healthy and supple and to help prevent cracking or flaking due to mechanical stress. In addition, NMF allows the coneocyte cells to balance the osmotic pressure exerted by the intracellular “cement” surrounding them.

Keeping the solute concentrations balanced is important for preventing both excessive water influx, as seen in the wrinkled skin after a long bath, and water efflux, which would cause the corneocytes to shrink.

Traditionally, the stratum corneum is thought of as nonviable tissue. While this is technically true, the stratum corneum is a dynamic structure in which numerous enzymes still function, and these enzymes require a certain amount of free, or liquid, water to perform. NMF-bound water provides much of this necessary water, and many of these enzymes are involved in the process of desquamation, breaking the various bonds and forces holding the corneocytes together in the most superficial layers of the skin. Research shows the activity of these desquamatory enzymes is affected by water levels within the tissue (Harding, 2000).

Reduction in, or the lack of, NMF has been correlated with various stratum corneum abnormalities that manifest clinically as areas of dry skin with scaling, flaking, or even fissuring and cracking. These conditions include atopic dermatitis, psoriasis, ichthyosis vulgaris, and xerosis. In atopic dermatitis, it has been shown that the amounts of NMF in the skin are often reduced (Palmer, 2006) while, in psoriatic skin and ichthyosis, NMF is essentially absent (Harding, 2000). Reduced NMF levels are also seen in more common skin conditions such as xerosis. Routine soap washing of the skin has been shown to remove NMF from the superficial layers of the stratum corneum. In fact, the outermost layers typically show reduced NMF levels, largely due to bathing or exposure to UV light. In addition, aging appears to dramatically reduce the amino acid content in the stratum corneum. Studies have shown a significant correlation between the hydration of the skin and its amino acid content (Horri, 1989). All of these conditions show characteristics of abnormal desquamation, with the accumulation of corneocytes resulting in the visible dryness, roughness, scaling, and flaking properties of dry skin (Harding, 2000).

The source of NMF was the subject of intensive research for a considerable time. Numerous studies on urocanic acid and PCA established that these compounds were derived from amino acids in the stratum corneum, which had been assumed to contain no active enzymes, as noted above. As a result of this research, it is now recognised that, while the stratum corneum is biologically dead, it is biochemically very active. Analysis of the amino acid composition of the stratum corneum eventually led to the discovery that NMF components were breakdown products resulting from the proteolysis of the filaggrin protein (Scott, 1982).

Filaggrin is a large, histidine rich protein localised in newly formed corneocytes, present in the corneocyte layer above the granular layer. The function of filaggrin is to aggregate filaments, and specifically to align epidermal and inner root sheath keratin filaments into highly ordered linear arrays, or macrofibrils.

Filaggrin has a high-molecular-weight precursor, profilaggrin, which originates in the keratohyalin granules of the granular layer. As the granular cells differentiate into cornified cells, profilaggrin is dephosphorylated and degraded into the highly basic, lower molecular weight filaggrin. It is at this stage that filaggrin works to aggregate filaments, catalysing the formation of disulphide bonds between the keratin fibres. These aggregated fibres form part of the envelope surrounding the cells entering the stratum corneum, allowing them to maintain the extremely flattened shape characteristic of corneocytes (Scott, 1982).

Filaggrin is subject to almost immediate proteolytic and degradative attack, once the keratin fibres have been formed. One of the first steps in this degradation process is the conversion of the filaggrin arginine residues to citrulline residues. This process increases the acidity of the filaggrin molecule, resulting in the loosening of the filaggrin/keratin complex and increasing the access of proteolytic enzymes. At this point, the filaggrin molecules are completely degraded into their respective amino acids and derivatives, which go to make up 70 to 100 percent of the free amino acids and their derivatives present in the stratum corneum (Scott, 1982).

The conversion of filaggrin to NMF occurs as the corneocytes are moving to the more superficial layers of the stratum corneum. The timing and exact depth in the stratum corneum of filaggrin processing is dependent on the water activity within the corneocyte and the external relative humidity. In a humid environment, where there are no drying effects, the hydrolysis of filaggrin occurs almost at the outermost surface. In low humidity, proteolysis occurs at deeper layers where NMF works to prevent desiccation of the skin (Harding, 2000). It has been demonstrated that occlusive patches applied to the skin can prevent filaggrin degradation altogether.

Conversion of filaggrin to NMF is also controlled by the water activity within the corneocyte, and only occurs within a narrow range—if the water activity is too high, filaggrin is stable, while if it is too low, the hydrolytic enzymes will be unable to function and degrade the filaggrin (Harding, 2000). Thus, the hydration status of the skin influences the degradation process of filaggrin.

Importantly, the creation of NMF creates substantial osmotic pressure within the corneocyte. Therefore, the degradation process does not occur until the corneocytes have matured and strengthened and migrated toward the more superficial layers of the stratum corneum, where the surrounding lipids and other extracellular components balance the resulting osmotic pressure (Harding, 2000).

NMF is generally considered to comprise the water-extractable material released by 30 minutes water treatment of acetone/ether treated stratum corneum (Jokura, 1995). The water extractable material is considered to be the total natural moisturising factor found within the stratum corneum. Typically, the composition of NMF is approximately: amino acids 48.3%; PCA 10.2%; uric acid 2.1%; lactic acid 10.1%; citric acid 7.9%; other organic acids 2%; urea 14%, and inorganic ions 5.2%. The inorganic ions which account for 5% of NMF include potassium, sodium and calcium. The calcium ions and potassium ions are important in terminal differentiation of epidermis and disappear after barrier perturbation, whereas the magnesium ions accelerate skin barrier recovery in the stratum corneum (Nakagawa, 2004). Sodium pyrrolidone carboxylic acid (PCA) and lactic acid are both highly hydroscopic and act as efficient humectants, with both accounting for approximately 10% of NMF. The largest percentage of NMF is the amino acids at 48%, with neutral amino acids accounting for 34.5%, acidic amino acids contributing 5% and basic amino acids making up the remaining 8%.

Serine is the largest free amino acid found within NMF and accounts for 36% of all free amino acids found in NMF. Glycine is the second largest free amino acid at 22%, followed by alanine attributing for 13% of free amino acids in NMF. Histidine (8%), ornithine (7%), citrulline (6%), arginine (6%), and proline (2%) are all also present within NMF.

While the importance of NMF in skin hydration has been understood by some skin researchers since the 1960s, and the relationship of NMF to filaggrin processing determined in the 1980s, the full significance of the association was only appreciated with the recent identification of filaggrin loss-of-function mutations.

Inherited loss-of-function mutations in the filaggrin gene (FLG) have been shown to cause moderate-to-severe ichthyosis vulgaris, and to predispose patients to atopic dermatitis, including early-onset atopic eczema that recurs or persists into adulthood. In atopic dermatitis, the levels of PCA, urocanic acid, and histidine have been shown to be correlated with the FLG genotype, being reduced in patients carrying various FLG mutations. Multiple mutations in the FLG gene have been identified; just two of these variants are carried by approximately nine percent of people of European origin, suggesting a prevalence of filaggrin mutations in certain populations. Patients carrying loss-of-function filaggrin mutations have significantly reduced levels of NMF in the stratum corneum at all depths. In addition, carriers of filaggrin mutations exhibit increased transepidermal water loss compared with non-carriers.

Filaggrin proteolysis abnormalities can occur in response to environmental factors. As noted above, low humidity impairs the ability of hydrolytic enzymes to break down filaggrin into NMF, thus generating skin surface dryness. In addition, UV radiation has been shown to impair the natural breakdown of filaggrin to its NMF components. Further, NMF levels in the skin decline with age, and this decline has been attributed to the decreased synthesis of profilaggrin, and a decline in barrier function, in the elderly.

As noted above, approximately one-third of water contained within the stratum corneum is bound, with the remainder being free water. Increasing the level of free water has no effect on the elasticity of the stratum corneum, and it is NMF-bound water that provides the skin with its elastic qualities. Replacing or replenishing the supply of NMF in the skin through the external application of moisturisers containing NMF has proven to be a successful approach for the treatment of xerotic skin (Weber, 2012).

Several NMF components have been used for decades in moisturising vehicles, without knowledge of why they have any effect. For example, urea has been included in moisturising creams as far back as 1943 (Harding, 2000). However, skin urea levels, which are now known to be reduced in patients with atopic dermatitis, and in elderly skin, were not measured in normal and atopic patients until 1966. Topical application of urea, or its precursor, arginine, has been shown to correct urea deficits. Lactate was first reported to be used in a moisturiser as a treatment for ichthyosis in 1946. It has been shown to improve and prevent the reappearance of symptoms of dry skin compared with lactate-free moisturisers. L-lactic acid and D,L-lactic acid appear to work by stimulating the synthesis of ceramides in the stratum corneum. PCA is the most prevalent single component of NMF, and has been shown to be reduced in the outermost layers of the skin as a consequence of soap washing and/or age. Topical application of PCA has been widely reported to alleviate the symptoms of dry skin (Harding, 2000).

Within the skin, water can move into the atmosphere from the stratum corneum by passive diffusion. This normal movement of water is known as transepidermal water loss (TEWL). This results as there is no absolute barrier to water permeation. In a healthy epidermis, the water content should be around 40% at the stratum granulosum/stratum corneum interface and 15%-25% at the skin's surface. Visible scaling of the skin occurs when the water content is around 10% or lower.

Practical Dermatology (July 2012, 24-26) mentions a tyrosine derivative, without disclosing the nature of the derivative, which had been found to significantly build volume in the dermal matrix over a relatively short period of time when applied topically. No further information is provided.

It has now, surprisingly, been found that unnatural, hygroscopic amino acids are useful to hydrate and otherwise enhance the moisture retention and uptake properties of skin and keratinaceous structures. These properties also enable these amino acids to serve as penetration enhancers that can act synergistically with other penetration enhancers.

Thus, in a first aspect, the present invention provides an unnatural, hygroscopic amino acid for use in enhancing hydration and/or the moisture retention and/or uptake properties of an external keratinaceous structure of an animal. A preferred such structure is the skin, but the amino acids of the invention may be used on nails, horn, hair and the eyes.

The present invention also provides the use of an unnatural, hygroscopic amino acid to enhance hydration and/or the moisture retention and/or uptake properties of an external keratinaceous structure of an animal.

The present invention further provides a method for enhancing the hydration and/or moisture retention and/or moisture uptake properties of an external keratinaceous structure of an animal, comprising administering an effective amount of an unnatural, hygroscopic amino acid to said skin.

A surprising finding is that individual amino acids of the present invention are capable of enhancing permeation, or percutaneous absorption, of substantially lipophilic drugs, with increasing effects observed for increasing lipophilicity. Conventional penetration enhancers of the art show the biggest effect on those drugs that do not penetrate the skin well. However, the enhancement effect of the amino acids of the present invention seems to increase with log P, such that the effect is not as great for those drugs with more hydrophilic properties, but increases with increasing lipophilicity.

As used herein, the term ‘drug’ refers to any pharmacologically active agent that it may be desired to administer topically or transdermally.

Thus, there is further provided the use of an unnatural amino acid as a penetration enhancer for a drug intended for topical administration. Exemplary such drugs include steroids and other molecules that are retained in the stratum corneum, and which stick thereto or bind to keratin, and those with a log P>3, but the penetration enhancing effect is applicable to all drugs for topical administration, with the preference being for those with a more lipophilic than hydrophilic nature.

Three preferred drug and amino acid combinations are: metronidazole and N-hydroxyserine; diclofenac diethylamine (DDEA) and N-hydroxyglycine; and acyclovir and L-homoserine. However, in general, the preferred amino acids of the present invention are also preferred as penetration enhancers, with advantageous effects being observed for most amino acids of the invention, especially those having an O/C ratio of at least 0.7. An O/C ratio of 1 or more is advantageous.

Preferred amino acids for use as penetration enhancers include N-hydroxyserine, N-hydroxyglycine, L-homoserine and α-hydroxyglycine.

Other suitable drugs are as follows:

Type Of Drug

| | |
|---|---|
| Local antipruritics | Crotamiton |
| | Doxepin hydrochloride |
| | Mesulphen |
| | Polidocanol |
| 1 LOCAL ANAESTHETICS | Amethocaine (Hydrochloride in solutions |
| | or creams, base in gels or ointments) |
| | Amylocaine (Hydrochloride) |
| | Benzocaine |
| | Bucricaine (hydrochloride) |
| | Butacaine Sulphate |
| | Butyl Aminobenzoate Picrate |
| | Cincocaine (base, hydrochloride or |
| | benzoate) |
| | Dimethisoquin Hydrochloride |
| | Dyclocaine Hydrochloride |
| | Ethyl Chloride |
| | Lidocaine |
| | Lignocaine |
| | Myrtecaine |
| | Oxethazaine (Oxetacaine) |
| | Prilocaine |
| | Propanocaine Hydrochloride |
| | Tetracaine |
| Antihistamines | Antazoline |
| | Chlorcyclizine Hydrochloride |
| | Dimethindene Maleate |
| | Diphenhydramine |
| | Histapyrrodine |
| | Isothipendyl Hydrochloride |
| | Mepyramine |
| | Mepyramine Maleate |
| | Tolpropamine Hydrochloride |
| | Tripelennamine Hydrochloride |
| | Triprolidine Hydrochloride |
| Corticosteroids | Alclometasone dipropionate |
| | Beclomethasone dipropionate |
| | Betamethasone valerate |
| | Clobetasol propionate |
| | Clobetasone butyrate |
| | Desoximetasone |
| | Diflucortolone valerate |
| | Fludroxycortide/Flurandrenolone |
| | Fluocinolone acetonide |
| | Hydrocortisone |
| | Hydrocortisone acetate |
| | Hydrocortisone butyrate |
| Topical preparations for | Calcipotriol |
| psoriasis | Coal tar |
| | Dithranol |
| | 5-Fluouracil |
| | Ciclosporin |
| | Fumeric acid |
| | Lonapalene |
| | Methotrexate |
| | Methoxsalen |
| | Salicylic acid |
| | Tacalcitol |
| | Tazarotene |
| Topical preparations for acne | Azelaic acid |
| | Benzoyl peroxide |
| | Dithiosalicylic acid |
| | Motretinide |
| | Resorcinol |
| Topical antibacterials for acne | Clindamycin |
| | Erythromycin |
| ‘Dermatological drugs’ | Becaplermin (Diabetic skin ulcers) |
| | Bentoquatum (prevents allergic contact |
| | dermatitis caused by poison ivy) |
| | Gamolenic acid |
| | Glycolic acid (Photodamaged skin) |
| | Hydroquinone/Mequinol (Depigmenting |
| | agents) |
| | Ichthammol |
| | Keluamid (seborrhoeic dermatitis) |
| | Lithium succinate |
| | Monobenzone (vitiligo) |
| | Polyphloroglucinol Phosphate (Treatment |
| | of wounds and pruritic skin disorders) |
| | Sodium pidolate (humectant, applied as |
| | cream/lotion for dry skin disorders) |
| | Sulphur (mild antifungal/antiseptic) |
| | Sulphurated Lime (For acne, scabies, |
| | seborrhoeic dermatitus) |
| | Sulphurated Potash (Acne) |
| | Minoxidil (hair growth) |
| Topical retinoids and related | Adapalene |
| preparations for acne | Isotretinoin |
| | Polyprenoic acid |
| | Tretinoin |
| Other topical preparations for | Nicotinamide |
| acne | |
| Topical antibacterials | Amphomycin |
| | Bacitracin/Bacitracin Zinc |
| | Bekanamycin Sulphate |
| | Chloramphenicol |
| | Chlorquinaldol |
| | Chlortetracycline |
| | Framycetin sulphate |
| | Fusidic Acid |
| | Halquinol |
| | Mupirocin |
| | Mupirocin |
| | Neomycin sulphate |
| | Polymyxins (Polymyxin B Sulphate) |
| | Silver sulphadiazine (sulfadiazine) |
| | Sulphanilamide |
| | Sulphasomidine |
| | Sulphathiazole (sulfathiazole) Sodium |
| Topical antifungals | (Benzoyl peroxide) |
| | Amorolfine |
| | Benzoic acid |
| | Bifonazole |
| | Bromochlorosalicylanilide |
| | Buclosamide |
| | Butenafine Hydrochloride |
| | Chlormidazole Hydrochloride |
| | Chlorphenesin |
| | Ciclopirox Olamine |
| | Clotrimazole |
| | Croconazole Hydrochloride |
| | Eberconazole |
| | Econazole nitrate |
| | Fenticlor |
| | Fenticonazole Nitrate |
| | Flutrimazole |
| | Haloprogin |
| | Ketoconazole |
| | Mepartricin |
| | Miconazole nitrate |
| | Naftifine Hydrochloride |
| | Natamycin |
| | Neticonazole Hydrochloride |
| | Nystatin |
| | Omoconazole Nitrate |
| | Oxiconazole Nitrate |
| | Pyrrolnitrin |
| | Sertaconazole Nitrate |
| | Sodium Propionate |
| | Sulbentine |
| | Sulconazole nitrate |
| | Sulconazole Nitrate |
| | Terbinafine |
| | Tioconazole |
| | Tolciclate |
| | Tolnaftate |
| | Triacetin |
| | Undecenoates/Undecanoic Acid |
| Antiviral preparations | 1-Docosanol |
| | Aciclovir |
| | Brivudine |
| | Edoxudine |
| | Ibacitabine |
| | Idoxuridine |
| | Idoxuridine in dimethyl sulfoxide |
| | Imiquimod |
| | Penciclovir |
| | Vidarabine |
| Parasiticidal preparations | Benzyl benzoate |
| | Carbaryl |
| | Malathion |
| | Permethrin |
| | Phenothrin |
| Preparations for minor cuts | Cetrimide |
| and abrasions | Collodion |
| | Magnesium sulphate |
| | Proflavine |
| Topical circulatory | Heparinoid |
| preparations | |
| Antiperspirants | Aluminium chloride |
| | Glycopyrronium bromide |
| Transdermal drugs | Scopolamine |
| | Buprenorphine |
| | Granisetron |
| | Nicotine |
| | Rivastigmine |
| | Nitroglycerin |
| | Methylphenidate |
| | Clonidine |
| | Testosterone |
| | Norethindrone acetate |
| | Fentanyl |
| | Selegiline |
| | Ethinyl estradiol |
| | Norelgestromin |
| | Rotigotine |
| | Estradiol |
| | Oxybutynin |

While skin is generally referred to hereinbelow, it will be understood that this term includes reference to any other keratinaceous structure, such as nail, as well as other external membranes, such as the cornea, unless otherwise apparent from the context.

As used herein, an unnatural amino acid is one that is either not synthesised by the host of the skin to be treated, or which is not associated with a dedicated host tRNA therefor. It is an advantage of such amino acids, especially those not synthesised by the host, that they are less subject to catabolism, such as by naturally occurring enzymes, so that they are retained in the skin for longer than naturally occurring amino acids, so that any moisturising or permeation enhancing effect may be prolonged.

The terms ‘moisturising’, ‘moisture retention’, and ‘moisture uptake’, and related terms, are used herein interchangeably when illustrating the present invention, and reference to one includes reference to the others, unless otherwise apparent from the context. Individually, the terms have specific meanings. The term ‘moisturising’ is an inclusive term, and indicates substances or conditions that lead to the balancing, or progress towards balancing, of moisture levels in dry skin. Enhanced ‘moisture retention’ indicates a reduced propensity of skin to allow water to escape, and ‘moisture retention’ indicates the propensity of skin to retain water. ‘Moisture uptake’ is the property of skin to absorb water from the environment, such as humid air. The term ‘hydration’ includes both the level of water in the skin as well as the process of water uptake into the skin, such as in moisture uptake, supra.

As used herein, the term ‘hygroscopic’ indicates an amino acid that is capable of absorbing and retaining moisture from the atmosphere at a relative humidity (RH) of ≦50%, and preferably 40%, or less, at 32° C.

For a compound to be hygroscopic it must be able to form a non-bonding association with water. Magnesium sulphate is very hygroscopic and forms non-bonding interactions between the oxygen atom in water and the magnesium atoms. Further investigation into what makes a compound hygroscopic lead to work performed on soil samples and aerosols. The most abundant free amino acids found within aerosols are glycine, serine and alanine. These are also the three most abundant free amino acids found within NMF. The hygroscopic properties of humic materials in atmospheric aerosol experiments lead to investigation between the hygroscopic properties observed and the chemical structure of humic substances (Sasaki, 2007). Through this work it was determined that compounds with a higher oxygen to carbon ratio (O/C) generally exhibited greater hygroscopic properties (Sasaki, 2007). For example, L-serine has 3 oxygen atoms and 3 carbon atoms, so that it has an O/C=1.0.

Amino acids of the present invention are able to deliquesce at 32° C. Preferably, amino acids of the present invention have a deliquescence relative humidity (DRH) of no greater than 80% at 32° C. Preferred amino acids of the present invention have a DRH of no greater than 80% at 32° C. and an O/C ratio of at least 0.7.

Naturally occurring amino acids are the L-amino acids in animals, and the preferred animals to be treated with the present invention are the mammals. Preferred mammals are those that have exposed, or hairless skin, whether wholly or in part, and particularly preferred are humans.

The unnatural amino acids will generally be D-amino acids, but L-amino acids not synthesised in the animal to be treated can include unusual L-amino acids, such as α-hydroxyglycine and L-homoserine.

Where amino acids of the invention are novel, then these are provided as aspects and embodiments of the invention.

The amino acids of the invention are any molecule that comprises a COOH group linked via one or two, and preferably one, carbon atom to an imide or, more preferably, an amine group. While it is preferred that the amino acids of the invention are in their free, zwitterionic form, they may also be provided in salt form in solution, or as ion pairs.

The amino acids of the invention may be applied to the skin in any suitable form, such as cream, lotion, gel, unguent, ointment, mousse, foam, solution, injection, suspension, colloidal system or spray (propellant or pump), either in a carrier comprising an aqueous component, such as one that may act as a solvent for the amino acid, or in a carrier comprising an organic vehicle capable of dissolving or entraining the amino acid. Such forms may alternatively, or further, include one or more drugs for topical administration, and may further comprise any additional substances, such as film forming agents, antimicrobials, antioxidants, stabilisers, emulsifiers, sterlilants, thickeners, and colourants.

The application form may comprise the one amino acid of the invention, or may contain two or more amino acids of the invention. Regardless, the administration form may further comprise one or more additional moisturiser ingredients as taught in the art, and may comprise further amino acids, such as natural, hygroscopic amino acids, or natural and unnatural amino acids that are not as hygroscopic as the amino acids of the invention. A preferred naturally occurring amino acid is L-homoserine.

In one aspect, it is preferred to mimic NMF, by using one or more amino acid ingredients of NMF, preferably in amounts and/or ratios approximating those found in NMF. Amounts and proportions of amino acids in NMF are as detailed above.

It is further preferred to mimic NMF by including one or more non-amino acid ingredients of NMF, preferably in amounts and/or ratios approximating those found in NMF. These preferably include one or more salts, especially the sodium and potassium salts.

It is preferred that all ingredients of any administration form are pharmaceutically acceptable.

Preferred amino acids of the present invention have an O/C ratio of at least 1. A more preferred ratio is at least 1.5:1, and a ratio of 2:1, and also higher than 2:1 is preferred.

The following compounds are preferred compounds of the present invention:

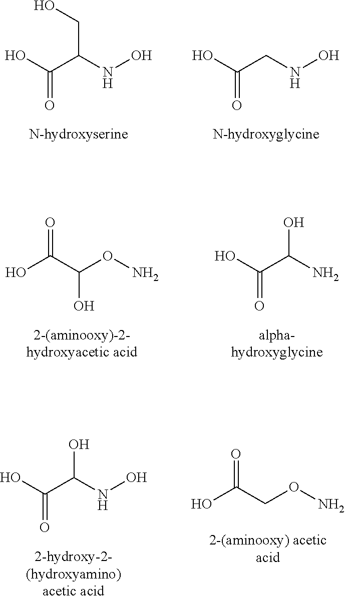 [SMILES: O=C(O)C(CO)NO ; O=C(O)CNO ; NOC(O)C(=O)O ; NC(O)C(=O)O ; O=C(O)C(O)NO ; NOCC(=O)O]

It will be appreciated that compositions comprising the amino acids of the present invention may be applied in preventative, or prophylactic capacity, particularly in cold weather, such as winter.

Compositions of the present invention may also be used for the treatment or prophylaxis of such conditions as inflammatory skin disease, atopic dermatitis, eczema, ichthyoses (dry skin conditions), winter xeroses, localised lichenifications, and eczematous episodes.

Compositions of the present invention are useful in the treatment of wounds, especially of topical membranes, preferably of the skin.

It will be appreciated that compositions of the present invention are particularly useful as cosmetic formulations. Such formulations may be for the enhancement of skin appearance, such as wrinkle treatment by plumping the skin, and skin elasticity, where the amino acids may be used in conjunction with collagen treatments, for example. Used on the nails, the cosmetic treatment may be used to soften the nails for cutting, or to hydrate the nails to help prevent chipping. In hair treatments, hydration may be used to increase suppleness of the hair and to help to prevent splitting.

It will be appreciated that the amino acids of the present invention may also be used in other applications that benefit from their skin moisturising properties. Thus, the present invention envisages the use of the amino acids of the invention as excipients in topical formulations. This may be to counteract the dehydrating effect of other excipients, such as ethanol, or simply as an emollient, or anti-dehydrating agent, or to enable enhanced drug absorption.

There is provided the use of amino acids of the present invention as anti-inflammatory agents, especially where a contributory factor in said inflammation is GM-CSF. Particularly preferred amino acids for such use are N-hydroxyserine, L-homoserine, N-hydroxyglycine, and combinations containing one or more thereof.

There is provided the use of amino acids of the present invention as anti-irritants. Particularly preferred amino acids for such use are N-hydroxyserine, L-homoserine, N-hydroxyglycine, and combinations containing one or more thereof.

The amino acids of the invention may find use as, or in, emollients.

The amino acids of the invention may be used in eye drops or other ocular formulation for the treatment of dry eye.

The amino acids of the invention may also be used to moisturise thickened skin, so as to facilitate callus removal, for example.

The amino acids of the invention may be used to help with nail and/or hoof softening.

The amino acids of the invention may also be used in conjunction with hair removal techniques.

Given the hydrative nature of compositions of the invention, they also find use in face packs.

The amino acids of the invention may also be used to hydrate the skin in the treatment of skin conditions including psoriasis, warts and verrucae, thereby permitting more effective drug delivery to the target site.

It will be appreciated that the amino acids of the invention may be applied to the skin before application of a barrier preparation, especially where the aim is to prevent drying out, such as desiccation, of the skin.

In general, the amino acids of the present invention find use in cosmetic preparations, especially those that may cause skin drying, and in those intended to enhance skin health and appearance, such as skin moisturisers and anti-wrinkle creams. Hair products, such as hair conditioners, also benefit.

The following synthetic route illustrates how amino acids of the present invention may be conjugated. Such conjugates may optionally be used in addition to, or in place of, amino acids of the invention in any uses as indicated herein, save where the skilled physician decides otherwise, or wherein a conjugate is less preferred, such as for reasons of speed of uptake into the stratum corneum for example.

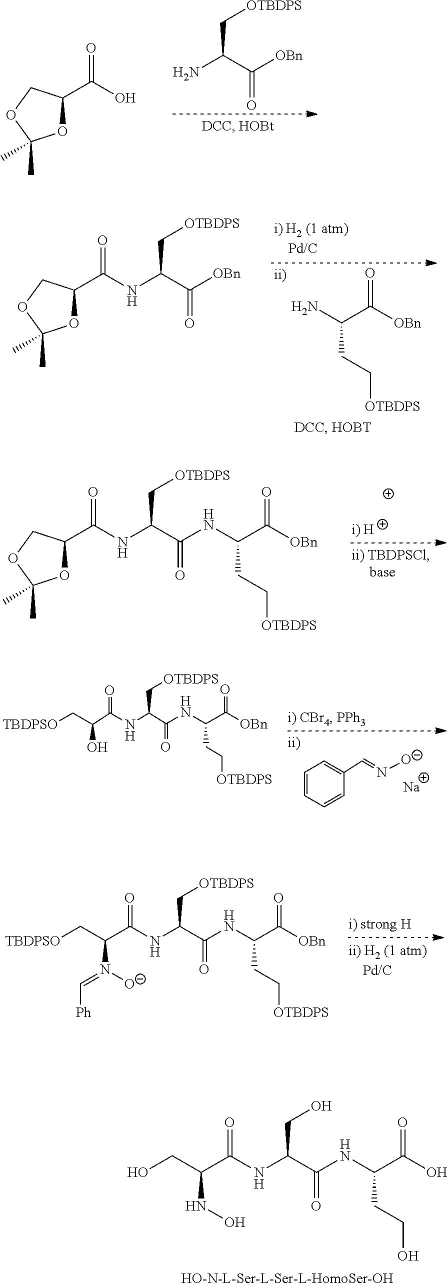

The invention will now be further illustrated with respect to the accompanying drawings, in which:

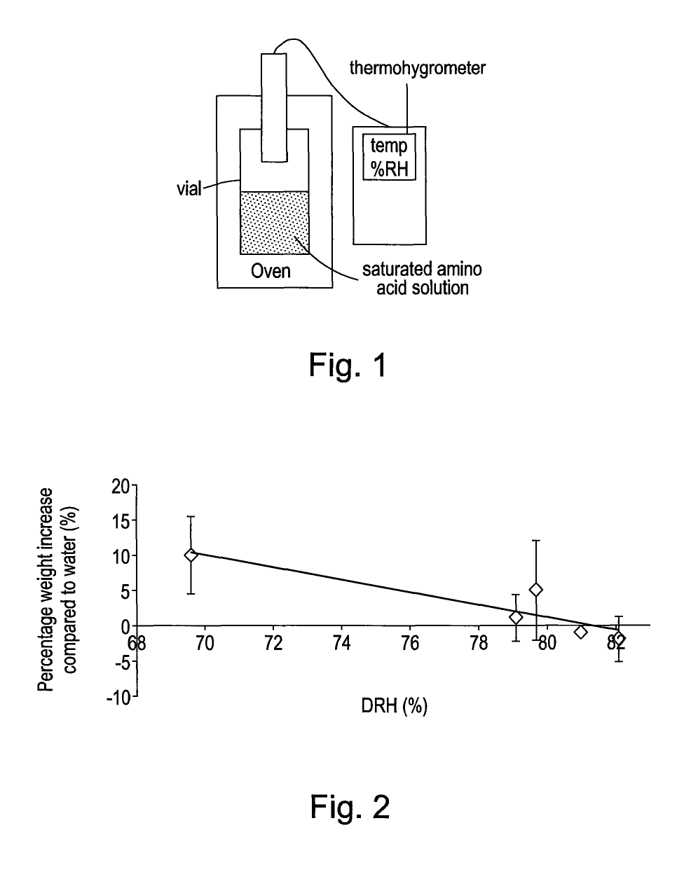

FIG. 1 shows an experimental set up for measuring the RH of a saturated solution;

FIG. 2 shows the average percentage weight increase after 24 h at 40% RH, compared to water against the DRH (deliquescence relative humidity) for the same compound;

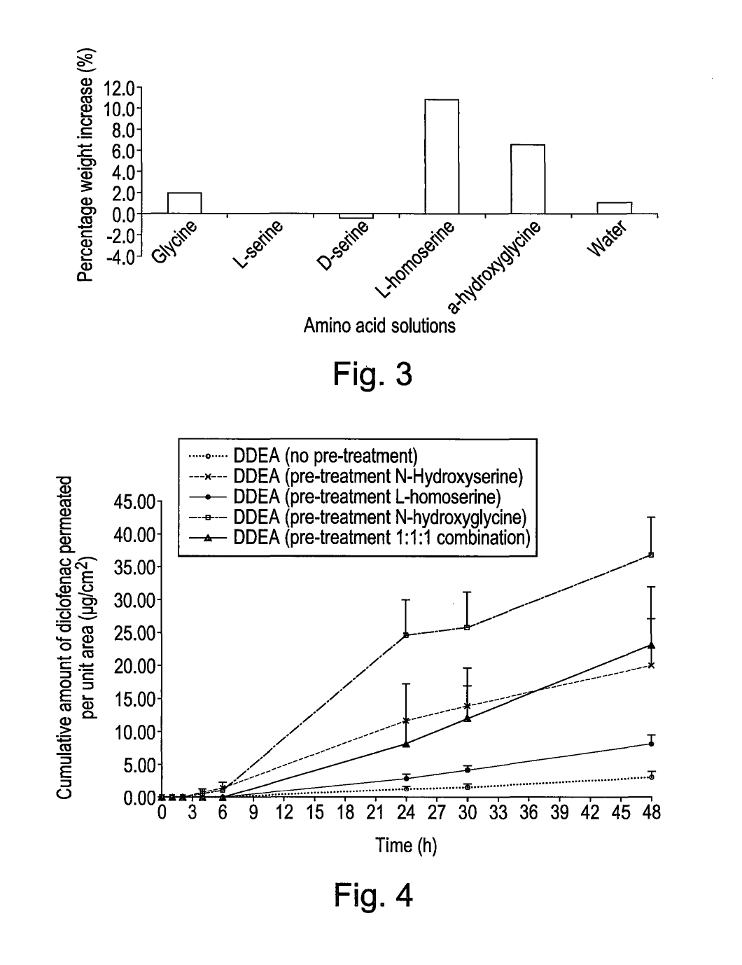

FIG. 3 shows the average percentage weight increase after 24 h at 40% RH, following 24 h of treatment with 1.33M amino acid solution, for each of the test amino acids;

FIG. 4 shows the cumulative permeation of diclofenac through epidermal membrane over a 48 h experimental period; each bar represents the average permeation+SEM, n=5-6;

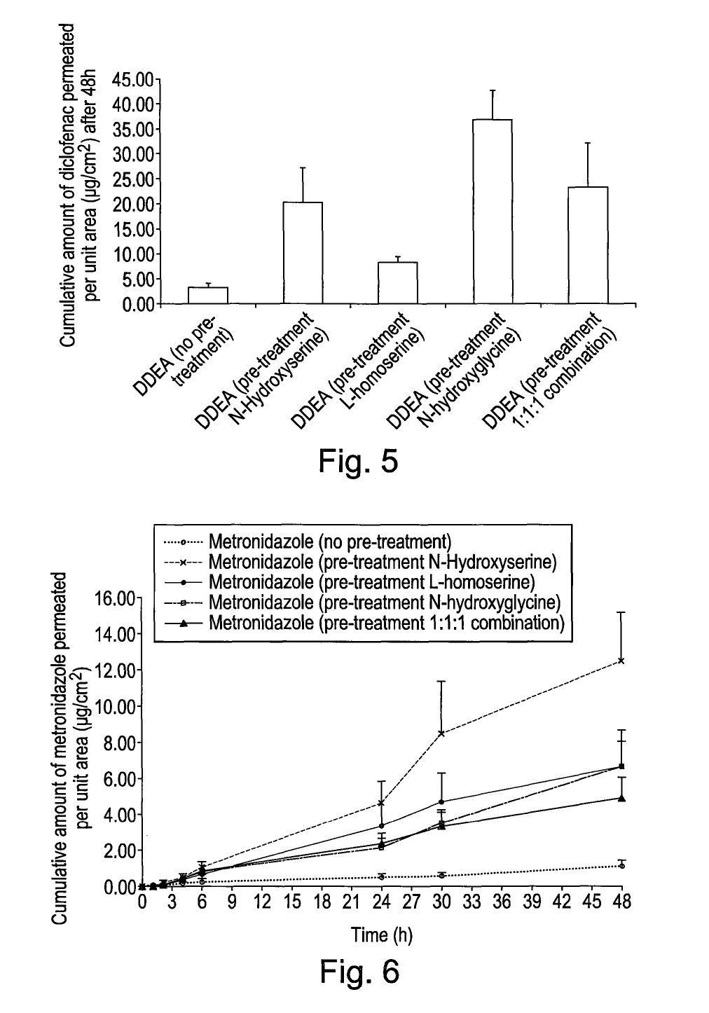

FIG. 5 shows the cumulative permeation of diclofenac through epidermal membrane after 48 h; each bar represents the average permeation+SEM, n=5-6;

FIG. 6 shows cumulative permeation of metronidazole through epidermal membrane over a 48 h experimental period; each bar represents the average permeation+SEM, n=5-6;

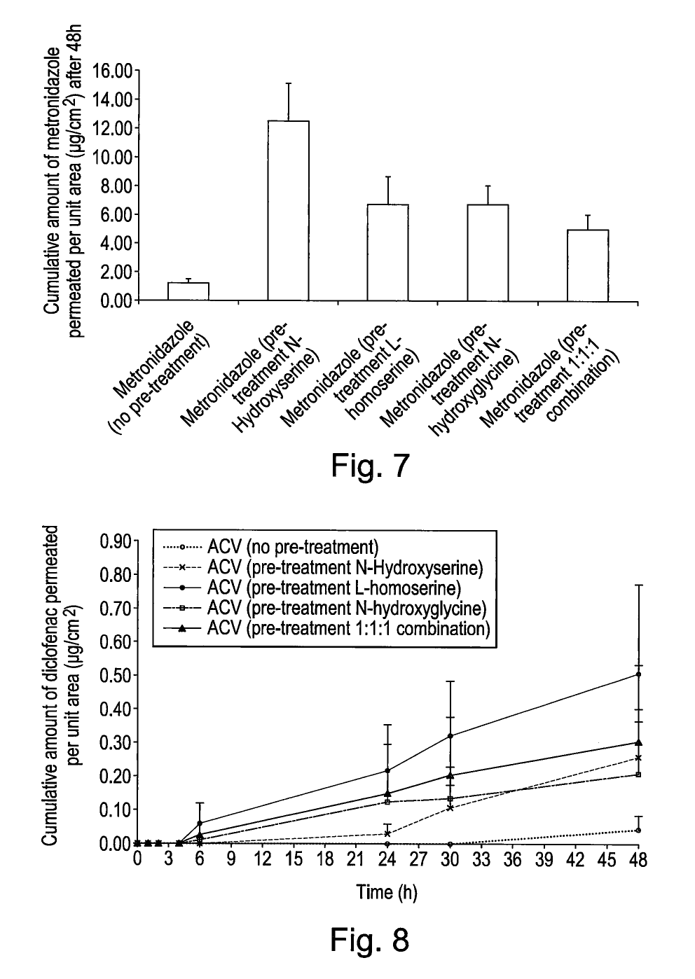

FIG. 7 shows cumulative permeation of metronidazole through epidermal membrane after 48 h; each bar represents the average permeation+SEM, n=5-6;

FIG. 8 shows cumulative permeation of Acyclovir through epidermal membrane over a 48 h experimental period; each bar represents the average permeation+SEM, n=5-6;

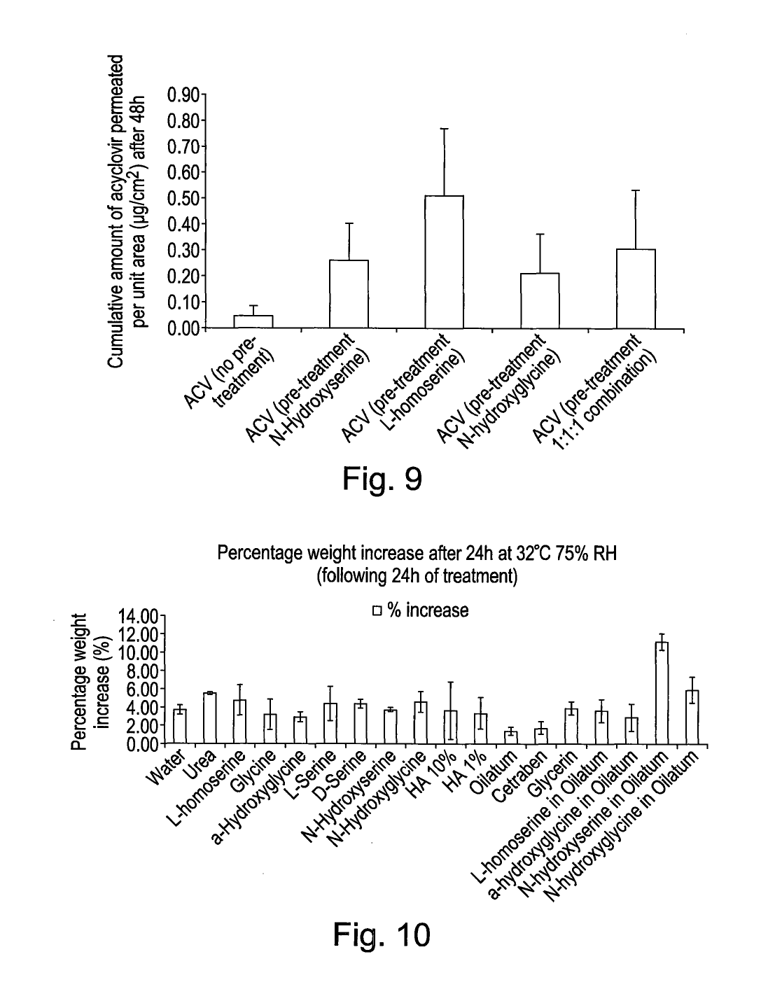

FIG. 9 shows cumulative permeation of Acyclovir through epidermal membrane after 48 h; each bar represents the average permeation+SEM, n=5-6;

FIG. 10 shows the average percentage weight increase after 24 h at 75% RH, following 24 h of treatment with 1.33M amino acid solution, for each of the test amino acids;

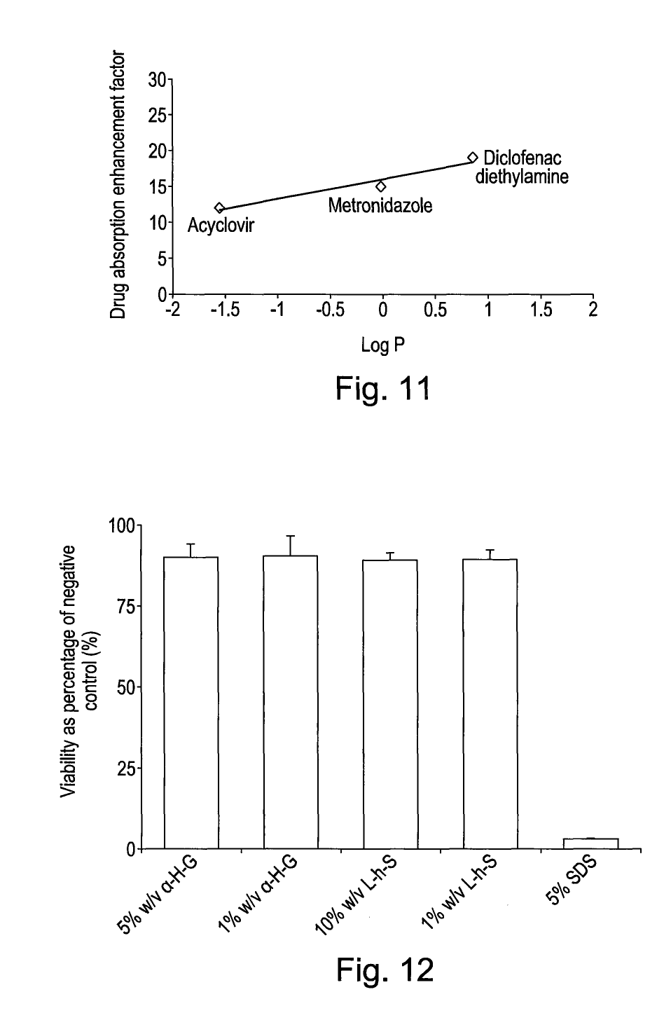

FIG. 11 shows the plot for drug absorption enhancement factor for 3 drugs;

FIG. 12 shows viability of RHE tissues post 42 minute exposure and 42 post exposure incubation; each point represents the mean tissue viability with error bars representing the range; n=4;

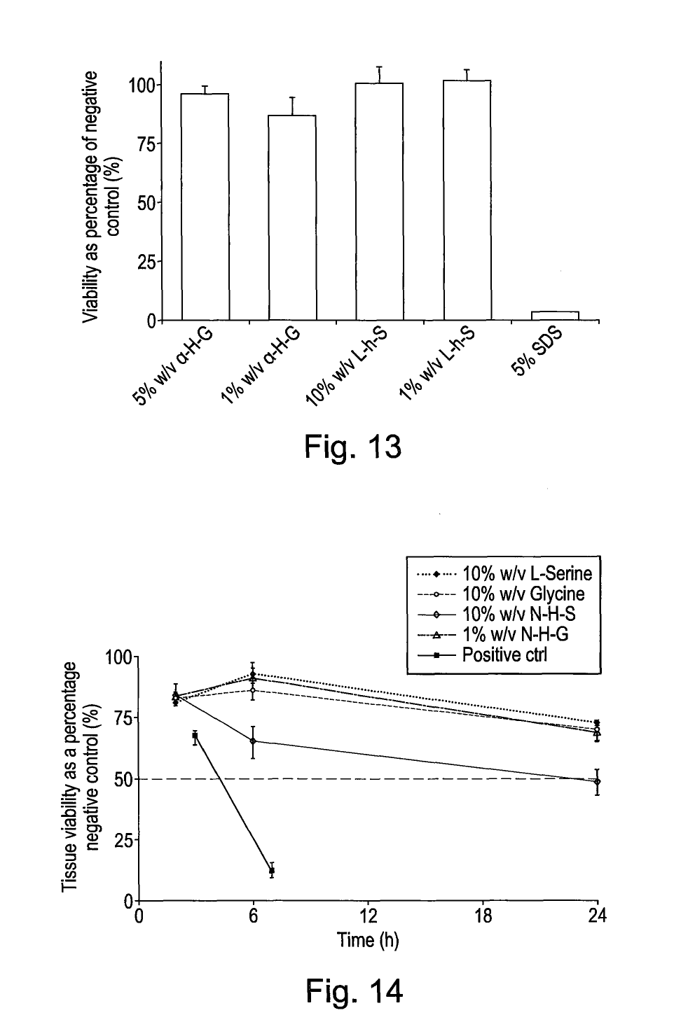

FIG. 13 shows viability of RHE tissues post 24 h exposure; each point represents the mean tissue viability with error bars representing the range; n=3;

FIG. 14 shows tissue viability after exposure to solutions of 10% w/v N-H-G, 1% w/v N-H-G, 10% w/v L-Serine and 10% w/v glycine at t=2, 6 and 24 h and positive control (1% w/v Triton™ X-100, t=3 and 7 h); each time point represents the mean tissue viability and error bars represent the range, n=3;

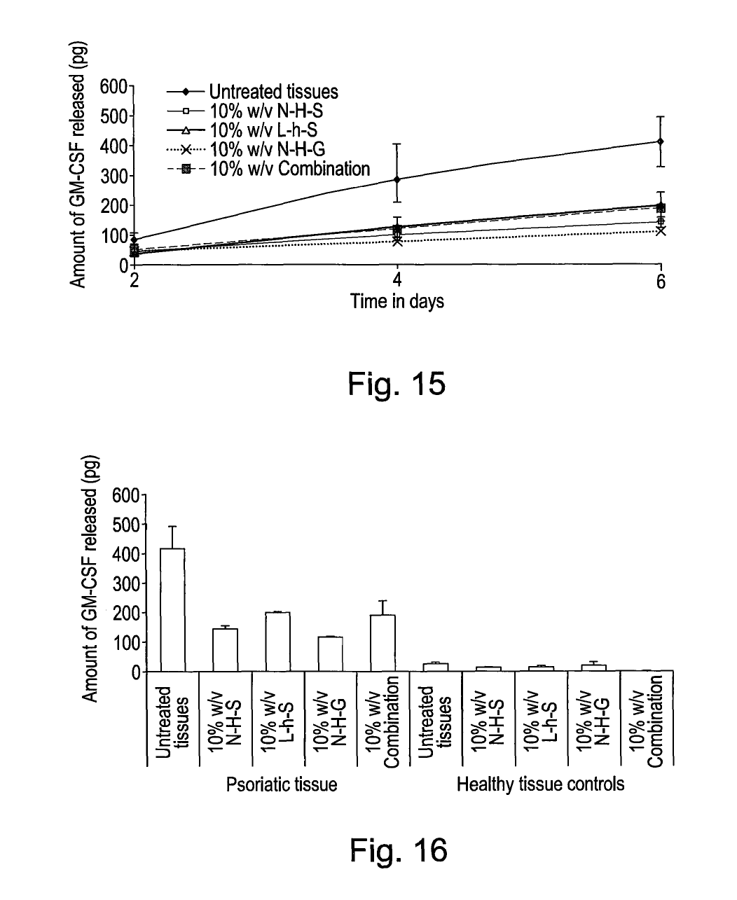

FIG. 15 shows release of GM-CSF from psoriasis tissues; each point represents the average cumulative release of GM-CSF over 6 day with measurements made at 2, 4, and 6 days with error bars representing the range n=2-3; and

FIG. 16 shows total release of GM-CSF. Each bar represents the total release of GM-CSF over 6 day from both psoriasis model and control RHE tissues; error bars represent the range n=2-3.

The present invention will now be illustrated by the following, non-limiting Examples.

### EXAMPLE 1

### Experimental

### 5-Bromopental

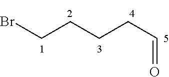

A solution of ethyl 5-bromopentanoate (3.9295 g, 18 mmol) in anhydrous DCM (100 mL) was stirred under a nitrogen atmosphere at −78° C., to which DIBAL-H (31 mL, 31 mmol, 1.7 equiv, 1M in hexanes) was added. The resultant orange solution was left stirring under a nitrogen atmosphere at −78° C., whilst the reaction was followed by TLC. After 8 h of stirring, the reaction was quenched with the addition of HCl (1M, 50 mL) and water (100 mL). The colourless mixture was removed from the nitrogen atmosphere and left stirring over night to gradually return to room temperature. The mixture was extracted with DCM (2×50 mL), dried over MgSO₄, filtered and concentrated in vacuo to afford the crude title product as a pale yellow oil (2.766 g, 80%, 21 mmol), which is used without further purification. R_{f}=0.30 diethyl ether/hexane (2/5).

¹H NMR (400 MHz, CDCl₃) 1.76-1.83 (2H, m, J=12.0 Hz, H-3), 1.87-1.94 (2H, m, J=12.0 Hz, H-2), 2.48-2.52 (2H, t, J=8.0 Hz, H-4), 3.41-3.44 (2H, t, J=8.0 Hz, H-1), 9.79 (1H, t, J=4.0 Hz, H-5) ppm.

¹³C NMR (400 MHz, CDCl₃) 20.6 (C-3), 31.9 (C-2), 33.0 (C-4), 42.9 (C-1), 201.8 (C-5) ppm. νₘₐₓ (FT IR, CCCl₃, KBr plates)/cm⁻¹ 2938 (C—H), 2725 (C—H aldehyde), 1721 (C═O), 1437 (C—H), 1390 (N—O), 1253, 1042, 913, 743.

### (E)-Ethyl-7-bromohept-2-enoate

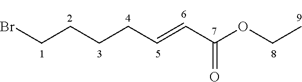

To a stirred suspension of crude 5-bromopental (2.766 g, 16 mmol) in DCM (100 mL) was added (carbethoxymethylene)triphenylphosphorane (8.098 g, 23 mmol, 1.4 equiv.) in one portion. The mixture was left stirring at room temperature for 22 h before being quenched with saturated ammonium chloride (50 mL). The mixture was extracted with DCM (2×50 mL) and the combined organic extracts were washed with water (50 mL). The organic layer was dried over MgSO₄, filtered and concentrated in vacuo to afford a crude yellow liquid (10.320 g). The crude product was then purified by column chromatography on silica gel eluting with diethyl ether/hexane (1/3) to afford the title product (2.723 g, 72%, 11.5 mmol); R_{f}=0.39 diethyl ether/hexane (1/3);

δ_{H} (400 MHz, CDCl₃) 1.27-.132 (3H, t, J=8.0 Hz H-7), 1.55-1.60 (2H, m, J=8.0 Hz, H-3), 2.02-1.06 (2H, m, J=16.0 Hz, H-2), 2.27-2.29 (2H, t, J=16.0 Hz, H-4), 4.16-4.22 (2H, q, J=32.0 Hz, H-1), 4.38-4.42 (2H, t, J=16.0 Hz, H-6) ppm.

δ_{C} (400 MHz, CDCl₃) 14.28 (C-9), 26.52 (C-3), 31.20 (C-2), 32.02 (C-4), 33.26 (C-8), 60.25 (C-1), 121.90 (C-6), 148.14 (C-5), 166.55 (C-7) ppm.

νₘₐₓ (FT IR, CCCl₃, KBr plates)/cm⁻¹ 2938 (C—H), 1714 (C═O), 1654 (C═C), 1445, 1367 (N—O), 1266, 1185, 1134 (C—O), 1095, 1039, 979 (C—H), 913 (C—H), 848, 742.

### (E)-Ethyl-7-nitrohept-2-enoate

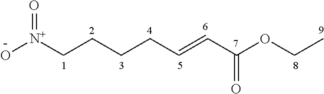

A solution of ethyl-7-bromohept-2-enoate (2.6486 g, 11.3 mmol) in DMF (13 mL) was stirred at 0° C., to which sodium nitrite (1.1657 g, 16.9 mmol, 1.5 equiv.) was added in one portion. The resultant solution was left stirring at 0° C., whilst the reaction was followed by TLC. After 8 h, TLC showed majority consumption of starting material. The reaction was added to ice cold water (20 mL) and extracted with diethyl ether (25 mL). The organic layer was then washed with brine (saturated 25 mL). The organic extract was dried over MgSO₄, filtered and concentrated in vacuo to give a yellow liquid (1.9390 g). The crude product was then purified by column chromatography on silica gel eluting with diethyl ether/hexane (1/4) to afford the title product (0.5283 g, 23%, 2.6 mmol);

R_{f}=0.29 diethyl ether/hexane (1/4);

δ_{H} (400 MHz, CDCl₃) 1.27-.131 (3H, t, J=8.0 Hz H-9), 1.54-1.62 (2H, quin, J=16.0 Hz, H-3), 2.00-2.08 (2H, quin, J=16.0 Hz, H-2), 2.25-2.30 (2H, q, J=16.0 Hz, H-4), 4.16-4.22 (2H, q, J=16.0 Hz, H-8), 4.38-4.42 (2H, t, J=8.0 Hz, H-1), 5.83-5.85 (1H, d, J=12.0 Hz, H-6), 6.88-6.95 (1H, dt, J=8.0 Hz, J′=16.0 Hz) ppm.

δ_{C} (400 MHz, CDCl₃) 14.28 (C-9), 26.52 (C-3), 31.20 (C-2), 32.02 (C-4), 33.26 (C-8), 60.25 (C-1), 121.90 (C-6), 148.14 (C-5), 166.55 (C-7) ppm.

νₘₐₓ (FT IR, CCCl₃, KBr plates)/cm⁻¹ 2936 (C—H), 2865, 1716 (C═O), 1644 (NO₂), 1445 (C—H, 1415, 1388, 1288 (C═C), 1229, 1170 (C—O), 1134, 1096, 1033, 912 (═C—H), 823, 734, 649.

### tert-butyl (2-chloro-2-oxoethyl)carbamate

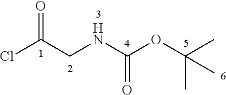

Boc-Gly (2.833 g, 16.2 mmol) was dissolved in anhydrous DCM (30 mL) under a nitrogen atmosphere at room temperature. The Boc-Gly solution was transferred to a solution of oxalyl chloride (3.10 mL, 2.2 equiv, 35.6 mmol) in anhydrous DMF (3 drops). The resultant solution was left stirring at room temperature under a nitrogen atmosphere, whilst the reaction was followed by TLC. After 3.5 h, TLC showed consumption of starting material. The mixture was concentrated in vacuo to yield the acid chloride (3.4634 g, 111%, 18 mmol).

R_{f}=0.15 diethyl ether/hexane (4/1);

¹H NMR (400 MHz, CDCl₃) 1.49 (9H, s, H-6), 3.69 (2H, s, H-2), 5.95 (1H, br s, H-3) ppm.

¹³C NMR (400 MHz, CDCl₃) 39.77 (C-6), 66.77 (C-2), 106.35 (C-5), 138.34 (C-4), 183.29 (C-1) ppm.

νₘₐₓ (FT IR, CCCl₃, KBr plates)/2989 (C—H), 1766 (C═O), 1742 (C═O), 1375, 1314, 1264, 1189, 1158, 1009 (C—N), 919, 852, 747 cm⁻¹.

### tert-butyl (2-((4R,5S)-4-methyl-2-oxo-5-phenyloxazolidin-3-yl)-2-oxoethyl)carbamate

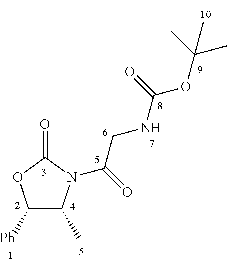 [SMILES: C[C@@H]1[C@H](c2ccccc2)OC(=O)N1C(=O)CNC(=O)OC(C)(C)C]

LiHMDS (2.5474 g, 15 mmol, 1.2 equiv) was added a cooled solution of (4R,5S)-(+)-4-methyl-5-phenyl-2-oxazolidone (2.2104 g, 12 mmol) in anhydrous THF (10 mL) at −78° C. The mixture was stirred for 30 mins before the addition of Boc-Gly-Cl (2.4634 g, 1.5 equiv, 18 mmol) in anhydrous THF (10 mL) before being warmed to room temperature. After 14 h of stirring at room temperature, the reaction was quenched with the addition of NH₄Cl (saturated 30 mL) as TLC showed consumption of the acid chloride. The mixture was extracted with ethyl acetate (50 mL). The organic layer was further extracted with sodium bicarbonate (sat, 50 mL). The organic layer was dried over MgSO₄, filtered and concentrated in vacuo to afford a deep blood red liquid (2.2964 g). The crude product was left to stand overnight at room temperature to yield a brown/red solid. A solution of diethyl ether/hexane (4/1) was added to the solid and filtered to yield the pure brown solid of the title product (1.2702 g, 32%, 3.8 mmol).

R_{f}=0.43 diethyl ether/hexane (4/1);

Mpt=106-111° C.

¹H NMR (400 MHz, CDCl₃) 0.00 (9H, s, H-10), 0.82 (3H, d, J=6 Hz, H-5), 1.55 (2H, s, H-6), 4.20 (1H, quin, J=8 Hz, H-4), 4.92 (1H, br s, H-7), 5.72 (1H, d, J=8 Hz, H-2), 7.52-7.26 (5H, m, H-1) ppm.

¹³C NMR (400 MHz, CDCl₃) 0.06 (C-10), 3.66 (C-5), 7.66 (C-6), 47.92 (C-4), 51.53 (C-2), 54.57 (C-9), 126-128 (C-1), 169.22 (C-3), 179.35 (C-8), 183.05 (C-5) ppm.

νₘₐₓ (FT IR, CCCl₃, KBr plates)/3200 (N—H), 1754 (C═O), 1500 (C═O amide), 913 (C═C bending), 744 cm⁻¹.

### (4R,5S)-3-(2-bromoacetyl)-4-methyl-5-phenyloxazolidin-2-one

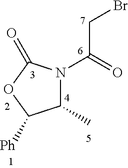 [SMILES: C[C@@H]1[C@H](c2ccccc2)OC(=O)N1C(=O)CBr]

n-BuLi (1 mL, 10 mmol, 1.1 equiv.) was added to a stirred solution of the oxazolidin-2-one (1.5930 g, 9 mmol, 1.0 equiv.) in dry THF (8 mL) at −78° C. under nitrogen. After 15 min, the bromoacetyl bromide (0.57 mL, 12 mmol, 1.3 equiv.) was added dropwise as a solution in THF (9.6 mL) and stirred at −78° C. for 15 min before being warmed to ambient temperature. After 2 h, the reaction was quenched with NH₄Cl (saturated 15 mL) and acetic acid (5 mL), extracted with EtOAc (20 mL), washed with NaHCO₃ (saturated 20 mL) and brine (saturated 20 mL), dried over MgSO₄ and concentrated in vacuo. The crude product was purified by flash column chromatography on silica gel eluting with EtOAc/hexane (3/17) to afford the title product (0.2593 g, 10%, 0.9 mmol).

R_{f}=0.41 EtOAc/hexane (3/17); ¹H NMR (400 MHz, CDCl₃) 0.93-0.94 (3H, d, J=4.0 Hz, H-5), 4.51-4.59 (2H, q, J=20.0 Hz, H-7), 4.77-4.83 (1H, q, J=12.0 Hz, H-4), 5.74-5.76 (1H, d, J=8 Hz, H-2), 7.26-7.46 (5H, m, H-1) ppm.

¹³C NMR (400 MHz, CDCl₃) 14.29 (C-5), 28.23 (C-7), 55.24 (C-4), 79.53 (C-2), 125.64 (C-1), 129.03 (C-1), 134.59 (C-3), 150.20 (C-6) ppm.

νₘₐₓ (FT IR, CCCl₃, KBr plates)/2964 (C—H), 2917, 2849, 1776 (C═O), 1700 (C═O), 1496 (Ph), 1455, 1415, 1340 (C—N), 1317, 1260, 1217, 1196 (C—O), 1169, 1146, 1120, 1089, 1066, 1037, 1001, 990, 970 911, 883, 803, 765, 730, 700, 661, 639, 618 cm⁻¹.

### (4R,5S)-3-(dibenzylglycyl)-4-methyl-5-phenyloxazolidin-2-one

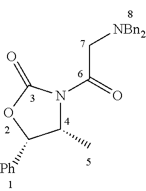

Dibenzylamine (0.485 mL, 3.0 mmol, 2.2 equiv.) was added to a stirred solution of the N-acyl-oxazolidin-2-one (0.4081 g, 1.37 mmol, 1.0 equiv.) in anhydrous DCM (1 mL) at rt. The reaction mixture stirred for 18 h under nitrogen. The resulting mixture is partitioned between DCM (10 mL) and water (10 mL). The aqueous layer was washed with DCM (10 mL). The combined organic layers are washed with water (10 mL), dried over MgSO₄ and concentrated in vacuo. The crude product was purified by flash column chromatography on silica gel eluting with EtOAc/hexane (3/17) to afford the title product (0.38844 g, 73%, 1.0 mmol).

R_{f}=0.30 EtOAc/hexane (3/17);

¹H NMR (400 MHz, CDCl₃) 0.87-0.89 (3H, d, J=8.0 Hz, H-5), 3.83-3.91 (2H, q, J=20.0 Hz, H-7), 3.95 (2H, s, H-8), 4.64-4.70 (1H, q, J=12.0 Hz, H-4), 5.61-5.63 (1H, d, J=8 Hz, H-2), 7.23-7.42 (15H, m, H-1) ppm.

¹³C NMR (400 MHz, CDCl₃) 14.67 (C-5), 54.56 (C-7), 55.24 (C-8), 58.18 (C-4), 79.50 (C-2), 125.64-129.03 (C-1), 167.23 (C-3), 184.49 (C-6) ppm.

νₘₐₓ (FT IR, CCCl₃, KBr plates)/3027 (═C—H), 2360, 2341, 1790 (Ph), 1705 (C═O), 1494 (Ph), 1454 (C—H), 1367, 1344 (C—N), 1245, 1218, 1197 (C—O), 1150, 1120, 1089, 1067, 1028, 984, 954, 917, 791, 766, 747, 698, 668, 643 cm⁻¹.

### 2-Amino-2-hydroxyacetic acid

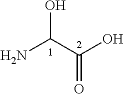

Ammonium acetate (9.4725 g, 0.1 mol, 2 equiv.) in ice-cold water (10 mL) was added to a stirred solution of glyoxylic acid monohydrate (4.6015 g, 0.05 mol, 1 equiv.) in ice cold water (10 mL). A white precipitate appeared within minutes of stirring. After 45 minutes of stirring and 2 h standing at 0° C., the reaction mixture was filtered. The white precipitate was washed with water (20 mL) and methanol (2×10 mL) to yield the crude title product (4.6951 g, 0.051 mol, 103%). The crude product (4.1697 g) was dried by a high vacuum pump for 5 hours to afford the title product (4.0571 g, 97%).

δ_{H} (400 MHz, D₂O) 4.93 (1H, s, H-1) ppm.

δ_{C} (400 MHz, D₂O) 26.81 (C-1), 175.83 (C-2) ppm.

νₘₐₓ (FT IR, CCCl₃, KBr plates)/cm⁻¹ 3056 (O—H, C—H), 1637 (C═O), 1561 (N—H), 1449, 1394, 1352, 1305 (C—O), 1194, 1129, 1071 (C—N), 881, 827, 616, 564, 536.

### N-Hydroxyglycine

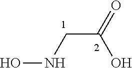

Glyoxylic acid (1.630 g, 22 mmol) and NH₃OH.HCl (1.1540 g, 22 mmol) are stirred in water (100 mL) at room temperature. 1M NaOH was used to raise the pH to 5 before the addition of sodium cyanohydridoborate (3.306 g, 52 mmol, 2.5 equiv). The reaction was left to stir for 48 h before 1M HCl was added drop wise to lower the pH to 1. The mixture was filtered and the filtrate was concentrated in vacuo. Water was added to the white residue and again the mixture was concentrated in vacuo to yield a white solid (7.7289 g). The white residue was taken up in water and passed through amberlite 200 C sodium. The product was removed from the amberlite by addition of 2% ammonia solution. The filtrate is concentrated in vacuo and recrystallised from hot ethanol to yield the yellow title product (0.6562 g, 33%, 7.2 mmol).

Melting point=137-139° C. δ_{H} (400 MHz, D₂O) 1.18 (1H, br s, N—H), 3.62 (2H, br s, H-1) ppm. δ_{C} (400 MHz, D₂O) 17.72 (C-1), 185.00 (C-2) ppm.

### Benzaldoxime

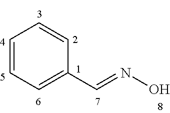

Benzaldehyde (2.00 mL, 20 mmol) was stirred in a mixture of ice:water:ethanol (2:1:1, 20 mL) at room temperature. Hydroxylamine hydrochloride (1.3860 g, 20 mmol) was added to the stirred suspension followed by 50% aqueous sodium hydroxide (4 mL, 40 mmol), while keeping the temperature below 30° C. After stirring for 1 hour at room temperature, the mixture was extracted with diethyl ether (2×25 mL). The aqueous extract was acidified to pH 6 using conc. HCl, while keeping the temperature below 30° C., before again being extracted with diethyl ether (2×25 mL). The combined organic extracts were dried over MgSO₄, filtered and concentrated in vacuo to yield a colourless liquid (2.4246 g, 100%, 20 mmol). δ_{H} (400 MHz, CDCl₃) 7.38 (2H, m, H-5, H-3), 7.58 (2H, m, H-2, H-6), 7.88 (1H, t, J=1.4 Hz, H-4), 8.15 (1H, s, H-7) ppm. δ_{C} (400 MHz, CDCl₃) 127.0 (C-5 and C-3), 128.8 (C-6 and C-2), 130.0 (C-4), 131.8 (C-1), 150.4 (C-7) ppm. νₘₐₓ (FT IR, CCCl₃, KBr plates)/cm⁻¹ 3280, 3061 (C—H), 3028 (C—H), 2985 (C—H), 2898, 2358, 2341, 1896, 1810, 1697 (C═N), 1631 (N—H), 1598 (C═C), 1577 (C═C), 1492 (N—O), 1443, 1303, 1288, 1209, 1176, 1158, 1102, 1074, 946, 868, 752, 702, 644. m/z (FTMS+ESI) found 122.0597 ([M+H]+, 100%) C₇H₈NO requires 122.0600.

### 2-bromo-3-hydroxypropanoic acid

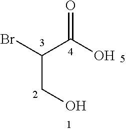

Potassium bromide (350.634 g, 2.94 mol) and L-serine (100.0 g, 0.95 mol) were stirred in 2.5M aqueous sulphuric acid (1.8 L) at 0° C. Sodium nitrite (92.0 g, 1.33 mol) was added slowly to ensure the temperature remained below 5° C. Half the sodium nitrite was added over the first 8 h, the mixture was left stirring over night before the remaining sodium nitrite was added over the next 30 mins. After stirring for 2.5 hours at 0° C., the mixture was extracted with diethyl ether (3×250 mL). The organic extracts are combined, dried over MgSO₄, filtered and concentrated in vacuo. The residue was then further concentrated on the high vac over 8 h to yield the title product (154.6969 g, 96%, 0.915 mol). δ_{H} (400 MHz, CDCl₃) 3.97-4.09 (2H, ddd, J=5.2 Hz, J′=12.1 Hz, J″=25.8 Hz, H-2), 4.40 (1H, t, J=5.3 Hz, H-3) ppm. δ_{C} (400 MHz, CDCl₃) 43.8 (C-2), 63.7 (C-3), 172.8 (C-4) ppm. νₘₐₓ (FT IR, CCCl₃, KBr plates)/cm⁻¹3407 (O—H), 2931 (C—H), 2355, 1722, 1615 (C═O), 1452, 1398, 1290, 1269, 1245, 1191 (C—O), 1160 (C—O), 1068, 1026, 909.

### Ethyl 2-bromo-3-hydroxypropanoate

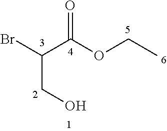

Concentrated sulphuric acid (2.8 mL, 0.003 mL per mmol) was added slowly to a stirred mixture of 2-bromo-3-hydroxypropanoic acid (154.6969 g, 0.92 mol) in absolute ethanol (1.83 L, 2 mL per mmol) before heating under reflux for 1.5 h. The mixture was cooled and ice cold water (1.83 L) was added and extracted with diethyl ether (2×1.8 mL). The organic extracts are combined and washed with ice cold water (1.8 L), 5% M aqueous sodium carbonate (2×1.8 L) and saturated brine (1.8 L). The organic extract was dried over magnesium sulphate, filtered and concentrated in vacuo to yield the title product (179.27 g, 99.5%, 0.91 mol). δ_{H} (400 MHz, CDCl₃) 1.30 (3H, t, J=6.8 Hz, H-6), 2.41 (1H, br s, H-1), 3.92-4.07 (2H, ddd, J=7.6 Hz, J′=12.0 Hz, J″=40.0 Hz, H-2), 4.25 (2H, J=7.2 Hz, H-5), 4.30 (1H, t, J=1.6 Hz, H-3) ppm. δ_{C} (400 MHz, CDCl₃) 13.92 (C-6), 44.61 (C-3), 62.43 (C-5), 63.85 (C-2), 166.7 (C-4) ppm. νₘₐₓ (FT IR, CCCl₃, KBr plates)/cm⁻¹ 3434 (O—H), 2987 (C—H), 2939 (C—H), 1736 (C═O), 1464, 1373, 1298, 1269, 1244, 1184, 1152, 1098 (C—O), 1080 (C—O), 1041 (C—O), 951, 857, 797, 678, 615.

### N-hydroxyglycine (alternative synthesis)

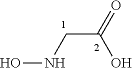

Sodium (0.4626 g, 0.02 mol) was added to a stirred mixture of benzaldoxime (2.3999 g, 0.02 mol) in absolute ethanol (40 mL). Ethyl bromoacetate (2.44 mL, 0.022 mol, 1.1 equiv.) was added and the mixture was stirred until the pH reached 7 which took 3 h. The mixture was filtered and the solid was washed with chloroform (2×40 mL). The combined filtrate was concentrated in vacuo. The residue was taken up in diethyl ether (50 mL) and placed in the fridge overnight. The mixture was filtered, washed with cold diethyl ether and the solid was dried under suction (3.1580 g). The nitrone (1.5 g) solid was stirred in conc HCl (20 mL) and heated under reflux for 0.5 h. The mixture was concentrated in vacuo. The residue was taken up in water and the pH was raised to 6 using ammonium hydroxide solution. The mixture was cooled in the fridge for 48 h, filtered and the solid was recrystallized from hot aqueous ethanol (75%) to yield (0.8095 g, 8.90 mmol, 76%). Melting point=137-139° C. νₘₐₓ (FT IR, CCCl₃, KBr plates)/cm⁻¹ 3375, 3234, 3094, 2908, 1645, 1592, 1549, 1508, 1406, 1305, 1214, 1178.

### N-hydroxyserine

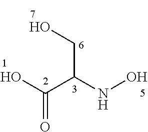

Sodium (0.4600 g, 0.02 mol) was added to a stirred mixture of benzaldoxime (2.4201 g, 0.02 mol) in absolute ethanol (40 mL). Ethyl 2-bromo-3-hydroxypropanoate (4.334 g, 0.022 mol, 1.1 equiv.) was added and the mixture was stirred until the pH reached 7 which took 3 h. The mixture was filtered and the solid was washed with chloroform (2×40 mL). The combined filtrate was concentrated in vacuo. The residue was taken up in diethyl ether (50 mL) and placed in the fridge overnight. The mixture was filtered, washed with cold diethyl ether and the solid was dried under suction (2.4131 g). The nitrone (1.5 g) solid was stirred in conc HCl (20 mL) and heated under reflux for 0.5 h. The mixture was concentrated in vacuo. The residue was taken up in water (10 mL) and the pH was raised to 6 using ammonium hydroxide solution. The mixture was cooled in the fridge for 48 h, filtered and the solid was recrystallized from hot aqueous ethanol (75%). Acetone was added to encourage the title product to drop out of solution to yield the title product (0.6724 g, 5.56 mmol, 88%). Melting point=159-163° C. δ_{H} (400 MHz, CDCl₃) 3.12 (1H, m, H-8), 3.56 (1H, m, H-8), 4.59 (1H, m, H-3) ppm. νₘₐₓ (FT IR, CCCl₃, KBr plates)/cm⁻¹ 3308 (O—H), 3032 (COOH), 2908 (CH), 1648 (N—H), 1591 (C═O), 1540 (N—O), 1511, 1402, 1306, 1229, 1174.

### Ethyl 2-bromo-2-hydroxyacetate

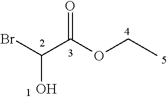

N-bromosuccimide (11.5343 g, 65 mmol) was stirred in carbon tetrachloride (80 mL) at 80° C. Dibenzyoyl peroxide (0.0521 g, 0.2 mmol) in ethyl glycolate (6.15 mL, 65 mmol) was added drop wise to the mixture before heating under reflux for 30 minutes, by which time the exothermic reaction had subsided. The mixture was cooled to room temperature, filtered and concentrated in vacuo. The residue was left until a precipitate formed, which took around 3 days. The mixture was filtered and washed with dichloromethane to yield a colourless solid (0.8544 g, 4.7 mmol, 7%). Melting point=107-110° C. δ_{H} (400 MHz, CDCl₃) 1.07 (3H, t, J=3.9 Hz, H-5), 2.66 (1H, br s, H-1), 3.44 (2H, q, J=7.1 Hz, H-4), 4.01 (1H, s, H-2) ppm. δ_{C} (400 MHz, CDCl₃) 16.7 (C-5), 57.3 (C-4), 86.0 (C-2), 172.9 (C-3) ppm. νₘₐₓ (FT IR, CCCl₃, KBr plates)/cm⁻¹ 3479 (O—H), 3406 (C—H), 1697 (C═O), 1614, 1439 (C—H), 1393, 1228 (C—O), 1191 (C—O), 1083 (C—O), 904, 808, 776, 719.

### EXAMPLE 2

### Experiment Summary

This experiment was to measure the deliquescence relative humidity (DRH) caused by a saturated amino acid solution in a sealed vessel at 32° C.; the lower the DRH, the greater the water holding capacity of the solution measured. Deliquescence relative humidity is the relative humidity at which the compound deliquesces, i.e. the relative humidity in which the compound absorbs so much water that it dissolves within the absorbed water.

### Equipment:

A diagrammatic representation of the equipment set-up is shown in FIG. 1, which shows an experimental set up for measuring the RH of a saturated solution.

The apparatus is maintained at 32° C. The temperature of 32° C. was chosen for being the temperature of outer stratum corneum. As the amino acids are to eventually be delivered to the outer layer of the skin, then the water holding capacity of these compounds at 32° C. is important.

### Methodology

The saturated solution is prepared as above by adding the amino acid to water (1 mL, 32° C.), whilst swirling. The total amino acid is weighed before adding to the water and the remaining amino acid so that the mass of compound used can be determined. The saturated solution (1 mL, 32° C.) is then transferred into the vial, again because of partial molar volume causing a change in volume by the addition of the amino acid to the water.

The temperature and % RH (% Relative Humidity) shown on the thermo-hygrometer are recorded every 30 minutes until the % RH remains constant. The temperature confirms that the temperature experienced by the sample is 32° C.

The results at 24 hours are summarised in Table 1, in which S.D. is standard deviation.

TABLE 1

| | | | O/C |
|---|---|---|---|
| Humectant | DRH | S.D. | Ratio |
| Urea 1.7 g | 75.03 | 6.04 | 1 |
| L-serine 0.95 g | 82.53 | 3.65 | 1 |
| D-serine 1.2 g | 82.13 | 8.82 | 1 |
| α-hydroxyglycine 0.35 g | 79.73 | 2.29 | 1.5 |
| L-homoserine 1.9 g | 71.37 | 4.72 | 0.75 |
| Glycine 1.1 g | 79.13 | 0.17 | 1 |
| Water (1 mL) | 98.73 | 1.79 | — |
| D-serine 0.95 g | 85.40 | 4.72 | 1 |
| Water (3 mL) | 100.00 | 0.00 | — |
| L-Proline | 44.6 | 2.6 | 0.4 |
| Zinc Nitrate | 45.8 | 2.6 | 0 |
| N-hydroxyglycine | 68.1 | 3.0 | 1.5 |
| N-hydroxyserine | 68.3 | 2.1 | 1.3 |
| Alanine | 83.1 | 11.0 | 0.67 |
| Threonine | 86.0 | 8.9 | 0.75 |
| Cysteine | 89.6 | 13.1 | 0.67 |
| Asparagine | 91.2 | 10.7 | 0.75 |
| Aspartic acid | 100.0 | 0.0 | 1 |

It can be seen that α-hydroxyglycine and L-homoserine, in these tests, demonstrated particularly advantageous levels of relative humidity. More particularly, amino acids of the present invention advantageously have a DRH of no greater than 80% at 32° C. In addition, amino acids of the present invention can also be seen to have an O/C ratio of at least 0.7.

From the above Table, from an average of 3 experiments, the relative humidity of water after 24 hr was 100%, as expected, serving as a control. L and D-serine had similar % RH, as expected, as they are enantiomers. Hydroxyglycine has an RH of 79.7%, better than the serines. L-homoserine had an RH at 24 hrs of 71.4%, thereby showing an excellent deliquescent relative humidity (DRH) measure.

### EXAMPLE 3

### Snake Study

### Protocol

All experiments were performed in triplicate (i.e. three separate solutions, three pieces of skin, one per solution). The skin used was from the dorsal side of Elaphe guttata, the American corn snake
A 1.33M concentration solution of each amino acid was made by stirring the amino acid (glycine 0.1008 g, L-serine 0.1395 g, D-serine 0.1400 g, L-homoserine 0.1582 g, α-hydroxy-glycine 0.1213 g) in water (1 mL).
Snake skin, from the top section (dorsal side) of the same donor, was cut in to ˜1 cm² pieces. Each piece was weighed and placed directly into one the of the amino acid solutions, one piece of snake skin per solution.
After 1 hour, the snake skin was removed from the solution and blotted dry on filter paper. The weight of the snake skin was recorded before the same snake skin was returned to the amino acid solution.
After an additional 23 hours (24 hours in total), the snake skin was again blotted dry on filter paper and weighed.
The snake skin, now having absorbed the test amino acid, was then placed in a vacuum desiccator over silica (0% RH) for 48 hours before being weighed. The skin was now dry, and contained the test amino acid.
The snake skin was then placed in a desiccator at 40% RH (controlled using a saturated solution of zinc nitrate), in order to establish how much water the skin was able to absorb from the atmosphere at this level of humidity.
Each skin sample was then reweighed.

Accompanying FIG. 2 shows the average percentage weight increase after 24 h at 40% RH, compared to water against the DRH (deliquescence relative humidity) for the same compound. FIG. 2 shows the DRH at 24 hrs after the skin was saturated with test amino acid, dried, and then exposed to 40% humidity.

Accompanying FIG. 3 shows the average percentage weight increase after 24 h at 40% RH, following 24 h of treatment with 1.33M amino acid solution, for each of the test amino acids.

Again, it can be seen that α-hydroxyglycine and L-homoserine, in these tests, demonstrated particularly advantageous properties.

### Protocol B

A 10% w/w concentration solution of each compound is made by stirring the compound (100 mg) in water (1 mL). Three separate solutions of each compound are made separately so that the experiment can be carried out in triplicate simultaneously. Where commercial creams are used, a sample of the cream (100 mg) is placed into vial.
Snake skin, from the top section of the same donor, is cut in to 1 cm² pieces. Each piece is weighed and placed directly into one of the compound solutions. One piece of snake skin per solution. The snake skin is placed directly onto the top of the solution so that only one side comes into contact with the solution. Care needs to be taken when placing the snake skin into the solution so that the skin lays flat on the solution surface.
After 1 hour, the snake is removed from the solution and blotted dry on filter paper. The weight of the snake skin is recorded before the same snake skin is returned to the same compound solution.
After an additional 23 hours (24 hours in total), the snake skin is again blotted dry on filter paper and weighed.
The snake skin is then placed in a vacuum desiccator over silica (0% relative humidity (RH)) for 48 hours before being weighed. The silica should be dried for 24 h before being placed into the vacuum desiccator.
The snake skin is then placed into a desiccator at a certain RH (40% RH is controlled using a saturated solution of zinc nitrate; 70% RH is controlled by a 1:1 NaCl:Na₂CO₃ saturated solution; 100% RH is controlled using water). Each chamber should be prepared 48 h prior to the snake skin sample being placed in to the chamber and the RH checked using a hygrometer.

The accompanying FIG. 10 shows the result the average percentage weight increase after 24 h at 70% RH, following 24 h of treatment with 10% w/w amino acid solution, for each of the test amino acids.

### EXAMPLE 4

The effect of pre-treatment of skin with amino acid formulations of the present invention to enhance the permeation of three compounds with different physicochemical properties was investigated. The three compounds were:
Acyclovir
Metronidazole and
Diclofenac diethylamine

### Methods

Acyclovir (ACV), metronidazole and diclofenac diethylamine (DDEA) were analysed using HPLC, using of a Waters Alliance Separations Module and Waters detector. The temperature of the column and samples were maintained at 45±2° C. and 5.0±2° C., respectively. A Kinetix™ C-18 (Phenomenex, USA) column (150 mm×4.6 mm 5 μm particle size) with a guard column (Phenomenex USA, C-18 4.0×3.0 mm) was employed as the stationary phase. The mobile phase was three part; mobile phase A, water; mobile phase B, methanol; and mobile phase C, 10 mM ammonium phosphate buffer pH 2.5. The mobile phase was run using a gradient flow (Table 2) with a flow rate of 0.8 mL/min. Samples were run for 12 minutes with an injection volume of 10 μL. Acyclovir, metronidazole and DDEA were processed at a wavelength of 276 nm, with approximate retention times of 4.7, 6.5, and 8.7 min, respectively. Calibration curves were constructed from a series of standards prepared by serial dilution in conjunction with separately prepared quality controls. Standards and QC's were diluted with receiver fluid (phosphate buffered saline). Data were recorded and analysed using Empower Pro³ Software.

TABLE 2
Flow gradient for analytical method.

| Time | % mobile phase A | % mobile phase B | % mobile phase C |
|---|---|---|---|
| 0.00 | 99.0 | 1.0 | 0.0 |
| 1.00 | 99.0 | 1.0 | 0.0 |
| 2.00 | 90.0 | 10.0 | 0.0 |
| 3.00 | 90.0 | 10.0 | 0.0 |
| 4.00 | 60.0 | 40.0 | 0.0 |
| 5.50 | 5.0 | 90.0 | 5.0 |
| 6.00 | 5.0 | 95.0 | 0.0 |
| 8.00 | 99.0 | 1.0 | 0.0 |
| 12.00 | 99.0 | 1.0 | 0.0 |

Solutions for pre-treatment of N-hydroxyserine, L-homoserine, N-hydroxyglycine and a combination of N-hydroxyserine, L-homoserine, N-hydroxyglycine in a 1:1:1 ratio were prepared in deionised water at 10% w/v.

Donor solutions of ACV, Metronidazole and DDEA were prepared by saturating the solvent system (50:50, PEG-400:water) with the API for ca. 16 h. The donor solutions were centrifuged prior to dosing in the in vitro permeation experiments.

Human epidermal membrane was prepared from skin post cosmetic reduction surgery (abdominoplasty). Full thickness skin was defrosted at ambient temperature until malleable. The subcutaneous fat was removed mechanically by blunt dissection. Upon removal of the fat, skin was immersed in hot deionised water (60±3° C.) for 45 s. The epidermal membrane (comprising the Stratum corneum and epidermis) was removed from the underlying dermis using a gloved finger and the dermis was discarded. The epidermal membrane was then floated (Stratum corneum side up) in deionised water onto filter paper. Excess water was removed from the surface and the tissue was mounted in Franz type diffusion cells. Each cell had an average surface area approximately 0.60 cm² and a volume of approximately 2.0 mL. The temperature of the water bath was set to maintain the surface temperature of the skin at 32° C. to represent skin in vivo.

Three experiments were performed, one for each API (Acyclovir, metronidazole and DDEA). During each experiment a total of 32 cells per API were prepared. The integrity of the epidermal membrane was assessed using electrical resistance to ensure the epidermal membrane was intact. The cells were each pre-treated with 100 μL of the 10% w/v solutions of N-Hydroxyserine (n=6), L-homoserine (n=6), N-hydroxyglycine (n=6) and the combination pre-treatment solution (n=6) overnight (ca. 16 h), n=6 cells were not pre-treated. In addition a blank (no pre-treatment) and a placebo cell (pre-treated with the combination pre-treatment solution) were also prepared to assess any potential interference with the analytical assay. Following the pre-treatment period the pre-treatment solution was removed from the surface of the epidermal membrane and the surface was dried. The lower receptor chamber was filled with receiver fluid (phosphate buffered saline) and the cells were dosed with a 6 mg dose (i.e. 10 mg/cm²) of each API saturated donor solution using a pre-calibrated positive displacement pipette with exception of the blank and placebo cells. Samples of receiver fluid (200 μL) were removed from the sampling arm at 0, 1, 2, 4, 6, 24, 30 and 48 h time points. After each sample was removed, an equal volume of pre-warmed receiver fluid was replaced. Samples were analysed using HPLC and the level of each API that had permeated was quantified.

### Results

Diclofenac permeation was observed to be notably higher from all cells that had been pre-treated with the amino acids of the invention (FIGS. 4 and 5). The highest amount of diclofenac permeated was observed following pre-treatment of the epidermal membrane with N-hydroxyglycine where at t=24 h, a maximal enhancement in diclofenac permeation was observed at 18.79 times that of the epidermal membrane that had no pre-treatment.

FIG. 4 shows the cumulative permeation of diclofenac through epidermal membrane over a 48 h experimental period. Each bar represents the average permeation+SEM, n=5-6.

FIG. 5 shows the cumulative permeation of diclofenac through epidermal membrane after 48 h. Each bar represents the average permeation+SEM, n=5-6.

TABLE 3
Mean cumulative permeation of diclofenac following no pre-treatment and pre-
treatment with novel amino acid solutions.

| | | pre-treatment N- | | pre-treatment L- | | pre-treatment N- | | pre-treatment 1:1:1 | |
|---|---|---|---|---|---|---|---|---|---|
| | no pre- | hydroxyserine | | homoserine | | hydroxyglycine | | combination | |
| | treatment | Mean | | Mean | | Mean | | Mean | |
| | Mean DDEA | DDEA | | DDEA | | DDEA | | DDEA | |
| | permeated | permeated | | permeated | | permeated | | permeated | |
| Time (h) | (μg/cm²) | (μg/cm²) | Mean ER | (μg/cm²) | Mean ER | (μg/cm²) | Mean ER | (μg/cm²) | Mean ER |
| 1.0 | 0.00 | 0.00 | n/a | 0.00 | n/a | 0.00 | n/a | 0.00 | n/a |
| 2.0 | 0.00 | 0.19 | n/a | 0.00 | n/a | 0.00 | n/a | 0.00 | n/a |
| 4.0 | 0.05 | 0.83 | 16.05 | 0.00 | 0.00 | 0.39 | 7.63 | 0.00 | 0.00 |
| 6.0 | 0.15 | 1.53 | 9.97 | 0.14 | 0.93 | 1.03 | 6.72 | 0.04 | 0.27 |
| 24.0 | 1.31 | 11.83 | 9.01 | 2.93 | 2.23 | 24.67 | 18.79 | 8.23 | 6.27 |
| 30.0 | 1.64 | 13.99 | 8.51 | 4.27 | 2.60 | 25.84 | 15.72 | 12.07 | 7.34 |
| 48.0 | 3.17 | 20.14 | 6.35 | 8.21 | 2.59 | 36.87 | 11.63 | 23.19 | 7.31 |

Enhancement ratio (ER) refers to the enhancement compared to diclofenac permeation with no pre-treatment.

In Table 4, metronidazole permeation was observed to be notably higher from all cells that had been pre-treated with the amino acids of the invention following the t=2 h time point (FIGS. 6 and 7). The highest amount of metronidazole permeated was observed following pre-treatment of the epidermal membrane with N-hydroxyserine where at t=30 h, a maximal enhancement in metronidazole permeation was observed at 14.72 times that of the epidermal membrane that had no pre-treatment.

FIG. 6 shows cumulative permeation of metronidazole through epidermal membrane over a 48 h experimental period. Each bar represents the average permeation+SEM, n=5-6.

FIG. 7 shows cumulative permeation of metronidazole through epidermal membrane after 48 h. Each bar represents the average permeation+SEM, n=5-6.

TABLE 4
Mean cumulative permeation of metronidazole following no pre-treatment and pre-
treatment with novel amino acid solutions.

| | no pre- | pre-treatment N- | | pre-treatment L- | | pre-treatment N- | | pre-treatment 1:1:1 | |
|---|---|---|---|---|---|---|---|---|---|
| | treatment | hydroxyserine | | homoserine | | hydroxyglycine | | combination | |
| | Mean | Mean | | Mean | | Mean | | Mean | |
| | metronidazole | metronidazole | | metronidazole | | metronidazole | | metronidazole | |
| | permeated | permeated | | permeated | | permeated | | permeated | |
| Time (h) | (μg/cm²) | (μg/cm²) | Mean ER | (μg/cm²) | Mean ER | (μg/cm²) | Mean ER | (μg/cm²) | Mean ER |
| 1.0 | 0.09 | 0.04 | 0.50 | 0.00 | 0 | 0.00 | 0 | 0.03 | 0.33 |
| 2.0 | 0.12 | 0.23 | 1.84 | 0.09 | 0.75 | 0.09 | 0.74 | 0.08 | 0.64 |
| 4.0 | 0.21 | 0.54 | 2.56 | 0.38 | 1.81 | 0.42 | 2.00 | 0.50 | 2.35 |
| 6.0 | 0.26 | 1.10 | 4.22 | 0.70 | 2.67 | 0.85 | 3.26 | 0.91 | 3.47 |
| 24.0 | 0.52 | 4.65 | 8.92 | 3.37 | 6.47 | 2.15 | 4.13 | 2.41 | 4.63 |
| 30.0 | 0.58 | 8.51 | 14.72 | 4.72 | 8.17 | 3.51 | 6.08 | 3.34 | 5.77 |
| 48.0 | 1.12 | 12.50 | 11.20 | 6.68 | 5.98 | 6.70 | 6.00 | 4.94 | 4.42 |

Enhancement ratio (ER) refers to the enhancement compared to metronidazole permeation with no pre-treatment.

In Table 5, Acyclovir permeation was observed in the receiver fluid earlier in cells that had been pre-treated with the amino acids of the invention (FIGS. 8 and 9). The highest amount of Acyclovir permeated was observed following pre-treatment of the epidermal membrane with L-homoserine where at t=48 h, a maximal enhancement in Acyclovir permeation was observed at 11.72 times that of the epidermal membrane that had no pre-treatment. Enhancement ratios prior to t=48 h could not be calculated due to the lack of Acyclovir permeation from the untreated skin prior to this point.

FIG. 8 shows cumulative permeation of Acyclovir through epidermal membrane over a 48 h experimental period. Each bar represents the average permeation+SEM, n=5-6.

FIG. 9 shows cumulative permeation of Acyclovir through epidermal membrane after 48 h. Each bar represents the average permeation+SEM, n=5-6.

TABLE 5
Mean cumulative permeation of Acyclovir following no pre-treatment and pre-
treatment with novel amino acid solutions.

| | no pre- | pre-treatment N- | | pre-treatment L- | | pre-treatment N- | | pre-treatment 1:1:1 | |
|---|---|---|---|---|---|---|---|---|---|
| | treatment | hydroxyserine | | homoserine | | hydroxyglycine | | combination | |
| | Mean | Mean | | Mean | | Mean | | Mean | |
| | Acyclovir | Acyclovir | | Acyclovir | | Acyclovir | | Acyclovir | |
| | permeated | permeated | | permeated | | permeated | | permeated | |
| Time (h) | (μg/cm²) | (μg/cm²) | Mean ER | (μg/cm²) | Mean ER | (μg/cm²) | Mean ER | (μg/cm²) | Mean ER |
| 1.0 | 0.00 | 0.00 | NC | 0.00 | NC | 0.00 | NC | 0.00 | NC |
| 2.0 | 0.00 | 0.00 | NC | 0.00 | NC | 0.00 | NC | 0.00 | NC |
| 4.0 | 0.00 | 0.00 | NC | 0.00 | NC | 0.00 | NC | 0.00 | NC |
| 6.0 | 0.00 | 0.00 | NC | 0.06 | NC | 0.01 | NC | 0.03 | NC |
| 24.0 | 0.00 | 0.03 | NC | 0.22 | NC | 0.12 | NC | 0.15 | NC |
| 30.0 | 0.00 | 0.11 | NC | 0.32 | NC | 0.14 | NC | 0.20 | NC |
| 48.0 | 0.04 | 0.26 | 5.99 | 0.51 | 11.72 | 0.21 | 4.82 | 0.30 | 7.00 |

Enhancement ratio (ER) refers to the enhancement compared to Acyclovir permeation with no pre-treatment.
*NC—Not calculated

### SUMMARY

Pre-treatment of the epidermal membrane with amino acids of the present invention enhances drug permeation thereover.

Different amino acids of the invention have differing enhancement effects depending on the physicochemical properties of the drugs.

The plot for drug absorption enhancement factor is shown in FIG. 11. From this it can clearly be seen that the enhancement increases with increasing Log P. In this regard, the enhancement factor and Log P are as follows:

| | ER | Log P |
|---|---|---|
| Diclofenac diethylamine | 19 | 0.85 |
| Metronidazole | 15 | −0.02 |
| ACV | 12 | −1.56 |

### EXAMPLE 5

### Water Uptake into Snake Skin Using Mixtures and Combinations

Combinations with Common Formulations and Bases

Using the protocol described in Example 3 with the only difference being that the snake skin was finally rehydrated over a solution at ˜75% RH (prepared using 1:1 NaCl:Na₂CO₃ saturated solution). All samples at 10% (except hyaluronic acid where one sample was used at 1% and vehicles were tested alone). The results are shown in FIG. 10.

From the results, it can be seen that a combination of an amino acid of the present invention with Oilatum is particularly beneficial in its moisturising ability.

### EXAMPLE 6

### RHE Irritation Assessment Method 1

### 1.1 Introduction

The purpose of this experiment was to investigate the potential of alpha-hydroxyglycine (α-H-G) and L-homoserine (L-h-S) to cause skin irritation. The method was based on the validated SOP for the “The SkinEthic Skin Irritation Test-42 bis assay” in accordance to OECD guideline Test No. 439: In Vitro Skin Irritation. The protocol is provided in Section 1.2. Further details on the method can be found in the SkinEthic™ RHE SOP, Version 2.1 (July 2009), SkinEthic skin irritation test-42 bis test method for the prediction of acute skin irritation of chemicals: 42 minutes application+42 hours post-incubation. Available at: [http://ecvam.jrc.ec.europa.eu]. In this protocol a tissue viability score of less than 50% of the negative control suggests the test solution is an irritant.

In addition, a further experiment was performed where the contact time of the test solution with the tissue was increased to 24 h. Following which point an MTT assay was used to determine the tissue viability and the test solutions potential to cause irritation.

### 1.2 the SkinEthic Skin Irritation Test—42 Bis Assay Protocol

1.2.1 RHE Irritation Testing Protocol

1.2.1.1.1 Pre-Incubation Step

The following steps were completed on receipt of the tissues:
(i) Wells of 6-well plates were filled with 1 mL growth culture medium.
(ii) Tissue were removed from their packaging, cleaned to remove the transport agarose and inspected for signs of damage; damaged tissues were discarded.
(iii) The tissue were transferred into the growth culture medium then incubated at 37° C., 5% CO₂ until application of the test solution.

1.2.1.1.2 Preparation of Phosphate Buffered Saline (PBS)
(i) PBS tablets (×5) were added to a 500 mL volumetric flask.
(ii) The volumetric flask from Step (i) was made to volume with deionised water (18.2 MΩ) and stirred using a PTFE magnetic stirrer until the PBS tablets are observed to dissolve.
(iii) The PBS was used as required

1.2.1.1.3 Preparation of MTT Solution
(i) 3-(4,5-Dimethylthiazol-2-yl)-2,5-diphenyltetrazolium bromide (MTT) (100.0±5.0 mg) was weighed into a 20 mL volumetric flask. The solution produced had a nominal MIT concentration of 5 mg/mL.
(ii) The volumetric flask form Step (i) was made up to volume with PBS.
(iii) A PTFE magnetic stirrer was added to the volumetric flask from Step (ii) and the solution was left to stir until fully dissolved.
(iv) The solution from Step (iii) was filter sterilised using a 0.2 μm filters directly into sterile tubes.
(v) This stock was protected from light and stored at −20° C. until required.
(vi) When required, the MTT solution was thawed and diluted with pre-warmed maintenance medium to achieve a concentration up to 1 mg/mL.

1.2.1.1.4 Preparation of Positive Control (5% SDS) Solution
(i) Sodium dodecyl sulphate (SDS, 100±5.0 mg) was weighed into a 20 mL volumetric flask and made to volume with deionised water (18.2 MΩ).
(ii) The volumetric flask from Step (i) was made to volume with deionised water (18.2 MΩ) and stirred using a PTFE magnetic stirrer until the SDS was fully dissolved.
(iii) The solution was filter sterilised using sterile 0.2 μm filter PES filters.

1.2.1.1.5 Application of 5 and 1% w/v α-H-G and 10 and 1% w/v L-h-S and Rinsing

1.2.1.1.6 Applications of 5 and 1% w/v α-H-G and 10 and 1% w/v L-h-S and Controls
(i) 16 μL±0.5 μL of the test solutions (5% w/v α-hydroxyglycine (α-H-G) in water, 1% w/v α-hydroxyglycine (α-H-G) in water, 10% w/v L-homoserine (L-h-S) in water, 1% w/v L-homoserine (L-h-S) in water) and negative (PBS) and positive (5% SDS) controls were dispensed onto the top of the epidermal tissue, using positive displacement pipette. The test solutions or controls were distributed over the surface epidermis using the tip of the pipette.
(ii) A nylon mesh was placed over the surface of the tissue using forceps and the plate lid was replaced.
(iii) The plates from Step (ii) were either retained in the laminar flow cabinet at room temperature for 42 minutes or incubated at 37° C., 5% CO₂ for 24 h.

1.2.1.1.7 Rinsing and Drying Steps
(i) Following treatment the nylon mesh was removed from the surface of the tissue.
(ii) The tissues were rinsed 25 times with PBS (1 mL) at a distance of 5-8 cm from the insert to remove all residual Test Item from the epidermal surface.
(iii) The inserts from Step (ii) were emptied and dried on sterile absorbent paper.
(iv) The surface of the Stratum corneum was dried with a cotton tip.
(v) The washed tissue from Step (iv) was transferred in a fresh 6 well culture plate prefilled with 2 mL of warmed growth culture medium,

1.2.1.1.8 Post Treatment Incubation: 42 Hours
(i) Post treatment the tissues were incubated at 37° C., 5% CO₂, 95% humidified atmosphere for 42 h.
(ii) At the end of the culture period the subnatant underneath the treated RHE tissues was retained.
(iii) The maintenance culture media was homogenised, by agitation at 300 RPM for 2 minutes.
(iv) The incubation medium was placed in centrifuge tubes and stored at −20° C. until required for analysis.

1.2.1.1.9 MTT Assay
(i) Required wells in 24-well plates were filled with 300 μL of the MTT solution and protected from light.
(ii) The tissues were removed from the post treatment incubation plates and excess culture media was removed with absorbent paper.
(iii) The treated tissues were transferred into wells in 24-well plates pre-filled with the MTT solution.
(iv) The plates were incubated for 3 h (+/−5 min) at 37° C., 5% CO₂, 95% humidified atmosphere.
(v) A new plate was filled with 800 μL of isopropanol and treated tissues were transferred into the plates. An additional 700 μL of isopropanol was added to the top of each tissue.
(vi) The plates were parafilmed to stop evaporation and protected from light using foil before being refrigerated (2-8° C.) overnight to extract.
(vii) Following extraction the tissue was pierced using a pipette tip in order to add the whole extraction solution into the well. The solution was made homogenous by mixing with a pipette.
(viii) 3×200 μL of extraction solution per well was pipetted into a 96 well plate and the optical density was measured at 570 nm using a μQuant spectrophotometer.

1.3 Results and Discussion

The results of the tissue viability assessments with 42 min and 24 h exposure are illustrated in FIG. 122 and Figure respectively. Tissues viability after exposure for all test solutions was observed to have remained over 50% of the negative control suggesting that the solutions (5% w/v α-H-G, 1% w/v α-H-G, 10% w/v L-h-S, 1% w/v L-h-S) are non-irritating.

FIG. 12 shows viability of RHE tissues post 42 minute exposure and 42 post exposure incubation. Each point represents the mean tissue viability with error bars representing the range; n=4.

FIG. 13 shows viability of RHE tissues post 24 h exposure. Each point represents the mean tissue viability with error bars representing the range; n=3.

### 2 RHE Irritation Assessment Method 2

### 2.1 Introduction

Method 1 was used to investigate the potential for alpha-hydroxylglycine (α-H-G) and L-homoserine (L-h-S) to cause skin irritation. The protocol is primarily designed and validated for single compounds rather than solutions. Therefore to provide further insight into the potential of N-hydroxyserine and N-hydroxylglycine to cause skin irritation a second protocol was investigated using Mattek's MTT effective time (ET-50) assay which is designed to test mixtures of compounds. In addition two commercially available amino acid related compound (L-serine and glycine) were selected and tested as comparators for N-hydroxyserine and N-hydroxylglycine.

Mattek's MTT effective time (ET-50) assay used a minimum of 3 exposure times (at each exposure time the tissue viability was determined by MTT assay) to construct a dose response curve to determine exposure time required for a chemical to reduce viability to 50% of the control (i.e. the ET-50 value). As a general guideline, the following groupings can be used in assigning expected in vivo irritancy responses based on the ET-50 results obtained:

| ET-50 (hrs) expected In vivo Irritancy | Example |
|---|---|
| <0.5 strong/severe, possible corrosive | conc. Nitric acid |
| 0.5-4 moderate | 1% Sodium Dodecyl Sulfate |
| 4-12 moderate to mild | 1% Triton X-100 |
| 12-24 very mild | Baby shampoo |
| 24 non-irritating | 10% Tween 20 |

### 2.2 MTT ET-50 Assay Protocol

The irritation assessment of the solutions was performed using the protocol described below. Each of the test solutions; N-hydroxyserine (10% w/v, N-H-S in water), N-hydroxylglycine (1% w/v, N-H-G in water), L-Serine (10% w/v, in water) and Glycine (10% w/v, in water) positive (1% v/v Triton X-100 in PBS) and negative (untreated) controls were applied to tissues at different dosing periods (2, 6 and 24 h for solutions, 5 h for negative control and 3 and 7 h for positive control, n=3).

### 2.2.1 Pre-Incubation Step of RHE Tissue

Tissues were stored at 2-8° C. on arrival, after which the following procedure was performed:
(i) Wells of 6-well plates were filled with pre-warmed (37° C.) assay medium (0.9 mL, supplied with the tissues) using an automatic pipette.
(ii) Tissue were removed from their packaging, cleaned to remove the transport agarose using cotton swabs and inspected for signs of damage. Damaged tissues were discarded.
(iii) The tissue were transferred into the assay medium and incubated at 37° C., 5% CO₂ until the application of the test solutions; N-hydroxyserine (10% w/v, N-H-S in water), N-hydroxylglycine (1% w/v, N-H-G in water), L-Serine (10% w/v, in water) and Glycine (10% w/v, in water) on the following day.

### 2.2.2 Preparation of the Working MTT Solution

The working MTT solution was prepared using the following procedure:
(i) MTT concentrate was removed from the freezer and allowed to thaw. The concentrate was diluted with the MTT diluent solution (1 mL of concentrate to 4 mL of MTT diluent solution).
(ii) MTT solution from Step (i) was centrifuged at a g-force of 300 for 5 min to remove any particulates.
(iii) The supernatant of the MTT solution from Step (ii) was stored in the dark at 4° C. for a maximum period of 24 h and used as required.

### 2.2.3 Applications of Solutions and Controls

(i) Prior to application of solutions and controls the pre-incubation media culture medium of the RHE tissues was removed from the 6 well plates replaced with fresh pre-warmed (37° C.) assay medium (0.9 mL, supplied with the tissues) using an automatic pipette.
(ii) Using a positive displacement pipette, the solutions N-hydroxyserine (10% w/v, N-H-S in water), N-hydroxylglycine (1% w/v, N-H-G in water), L-Serine (10% w/v, in water) and glycine (10% w/v in water) or controls (negative and positive control) were dispensed (100±0.5 μL) onto the top of the RHE tissue. The solutions or controls were then distributed over the surface of the RHE tissues using a sterile glass rod.
(iii) The lid was placed on the well plate containing the RHE tissue and the tissues were returned to the incubator (37° C., 5% CO₂) for the required dosing period (2, 6 and 24 h for the solutions; 3 and 7 h for positive control; 5 h for negative control).

### 2.2.4 MTT Assay

To determine the viability of the RHE tissue post application of the solutions and controls, the following procedure was used:
(i) The required wells in 24-well plates were filled with 300 μL MTT solution and protected from light.
(ii) The tissues were removed from the post treatment incubation plates and excess solutions and controls were removed with sterile cotton swabs.
(iii) The RHE tissue was subsequently rinsed with 1000 μL of PBS five times to remove the residual solution from the epidermal surface.
(iv) The tissue from Step (iii) was blotted dry with cotton swabs.
(v) The treated tissues from Step (iv) were transferred into wells in 24-well plates pre-filled with MTT solution (300 μL). The plates were incubated for 3 h (+/−5 min) at 37° C., 5% CO₂ and 95% humidified atmosphere.
(vi) The treated tissues were subsequently transferred into a new plate filled with 1 mL of extractant solution.
(vii) The plates were sealed using Parafilm® to stop evaporation and protected from light using foil before extract at 2-8° C. to minimise evaporation.
(viii) Following extraction the RHE tissue were removed from the wells and an additional 1 mL of extractant solution was added. Using a pipette the extraction solution was mixed and made visually homogenous.
(ix) The solution from Step (viii) was transferred in to n=3 wells (200 μL per well) using an automatic pipette of a 96 well plate and the optical density was measured at a wavelength of 570 nm.

### 2.3 Results and Discussion

The tissue viability assessments at 2, 6 and 24 h are illustrated in FIG. 14. The dose response curve demonstrates that, 1% w/v N-H-G, 10% w/v L-Serine and 10% w/v Glycine had no significant impact on the viability of the RHE tissues during the three dosing periods (t=2, 6 and 24 h). As such and in accordance with the ET-50 guidance documentation values 1% w/v N-H-G, 10% w/v L-H-S and 10% w/v Glycine would be considered as non-irritating.

An ET-50 at 22.8 h was calculated for 10% w/v N-H-S which would categorise 10% w/v N-H-S as a very mild irritant. Both of the commercially available amino acid related compounds (L-serine and glycine) were shown to be non-irritants.

FIG. 14 shows tissue viability after exposure to solutions of 10% w/v N-H-G, 1% w/v N-H-G, 10% w/v L-Serine and 10% w/v glycine at t=2, 6 and 24 h and positive control (1% w/v Triton™ X-100, t=3 and 7 h). Each time point represents the mean tissue viability and error bars represent the range, n=3.

### 3 In Vitro Assessment of Psoriatic Tissue. Method 3

### 3.1 Introduction

A human cell based in vitro psoriatic tissue model consisting of psoriatic fibroblasts and normal keratinocytes was used in this study. When compared to normal RHE tissues the psoriatic tissue model exhibits hyperproliferation of basal epithelial cells and increased expression of psoriasis markers compared to that of the normal RHE tissues (e.g. GM-CSF). GM-CSF is a key activator of the innate immune system involved in chronic inflammatory and autoimmune diseases including psoriasis (Löntz et al 1995), where macrophages, granulocytes, neutrophils, eosinophils and dendritic cells can contribute to tissue damage and disease progression (Krinner 2007). GM-CSF stimulates stem cells to produce granulocytes and other macrophages and subsequently activates these differentiated immune cells. GM-CSF has also been identified as an inflammatory mediator in autoimmune disorders with elevated levels of GM-CSF mRNA or protein being measured in a variety of inflammatory sites including in allergic and psoriatic patients, arthritic and asthmatic patients (Plater-Zyberk et al 2008). Numerous in vivo studies over the past few years have shown that blockade of GM-CSF via neutralising antibodies can prevent or even cure pro-inflammatory diseases in various models of inflammation including arthritis experimental autoimmune encephalitis, psoriasis (Schon et al 2000), and lung disease (Plater-Zyberk et al 2008). In addition treatment with recombinant GM-CSF was found to aggravate chronic inflammatory diseases, for example patients with chronic psoriasis receiving GM-CSF therapy have been shown to have exacerbated psoriatic lesions (Kelly et al 2007). Due to the importance of GM-CSF in psoriasis it was selected for analysis in this study.

### 3.2 Methods

In this investigation two types of RHE tissues were used, which were healthy and psoriatic tissue sourced from the MatTek Corporation (Ashland, Mass.). For simplicity the protocol below refers to both tissue types as RHE tissues and all media as assay medium as both tissues were treated in the same manner. The only exception was that two different types of media were required for the different types of tissue (healthy and psoriatic tissues). The healthy tissue controls were used in this experiment demonstrate that the levels of GM-CSF in the psoriatic tissue were evaluate as previously demonstrated by the manufacture.

3.2.1 Pre-Incubation Step of RHE Tissue

Tissues were stored at 2-8° C. on arrival, after which the following procedure was performed:
(i) Wells of 6-well plates were filled with pre-warmed (37° C.) assay medium (0.9 mL, supplied with the tissues) using an automatic pipette.
(ii) Tissue were removed from their packaging, cleaned to remove the transport agarose using cotton swabs and inspected for signs of damage. Damaged tissues were discarded.
(iii) The tissue were transferred into the growth culture medium and incubated at 37° C., 5% CO₂ until the application of the solutions on the following day.

### 3.2.2 Preparation MTT Solution

(i) 3-(4,5-Dimethylthiazol-2-yl)-2,5-diphenyltetrazolium bromide (MTT) (100.0±5.0 mg) was weighed into a grade A 20 mL volumetric flask. The solution produced had a nominal MTT concentration of 5 mg/mL.
(ii) The volumetric flask form Step (i) was made to volume with PBS.
(iii) A PTFE magnetic stirrer was added to the volumetric flask from Step (ii) and the solution was left to stir until fully dissolved.
(iv) The solution from Step (iii) was filter sterilised using a 0.2 μm filters directly into sterile tubes.
(v) This stock was protected from light and stored at −20° C. until required.
(vi) When required, the MTT solution was thawed and diluted with pre-warmed maintenance medium to achieve a concentration up to 1 mg/mL.

### 3.2.3 Tissue Setup and Application of Test Solutions

(i) At the end of the equilibration period, the media for each well was removed then the tissues were lifted and placed on culture stands and fresh pre-warmed (37° C.) assay medium (5 mL) was added to the well.
(ii) Using a positive displacement pipette, the test solutions 10% w/v, N-H-S(N-hydroxyserine) in water, 10% w/v, N-H-G (N-hydroxyglycine) in water, 10% w/v, L-h-S (L-homoserine) in water and 10% w/v, combination 1:1:1 (N-H-S:N-H-G: L-h-S) in water were dispensed (50±0.5 μL) onto the top of the RHE tissue. The solutions or controls were then distributed over the surface of the RHE tissues using a sterile glass rod.
(iii) The lid was placed on the well plate containing the RHE tissue and the tissues were returned to the incubator (37° C., 5% CO₂) 48 h between applications.

### 3.2.4 Media Change and Reapplication of Solutions

(i) Every 48 h culture media for the RHE tissues was removed from the 6 well plates and transferred into tubes stored (−20° C.) for cytokine analysis.
(ii) Fresh pre-warmed (37° C.) assay medium (5 mL) was added to each tissue using an automatic pipette.
(iii) From each treatment 1 tissue was removed for viability assessment.
(iv) The RHE tissues were subsequently rinsed with 100 μL of PBS to remove the residual solution from the epidermal surface.
(v) Using a positive displacement pipette, the solutions 10% w/v, N-H-S in water, 1% w/v, N-H-G in water, 10% w/v, L-h-S in water and 10% w/v, combination 1:1:1 (N-H-S:N-H-G:L-h-S) were dispensed (50±0.5 μL) onto the top of the RHE tissue. The solutions or controls were then distributed over the surface of the RHE tissues using a sterile glass rod.
(vi) The lid was placed on the well plate containing the RHE tissue and the tissues were returned to the incubator (37° C., 5% CO₂) 48 h between applications at t=0, 48 and 96 h.

3.2.5 MAT Assay

To determine the viability of the RHE tissue post application of the solutions and controls, the following procedure was used:
(i) The required wells in 24-well plates were filled with 300 μL MTT solution and protected from light.
(ii) The tissues were removed from the post treatment incubation plates and excess solutions and controls were removed with sterile cotton swabs.
(iii) The RHE tissue was subsequently rinsed with 1000 μL of PBS five times to remove the residual solution from the epidermal surface.
(iv) The tissue from Step (iii) was blotted dry with cotton swabs.
(v) The treated tissues from Step (iv) were transferred into wells in 24-well plates pre-filled with MTT solution (300 μL). The plates were incubated for 3 h (+/−5 min) at 37° C., 5% CO₂ and 95% humidified atmosphere.
(vi) The treated tissues were subsequently transferred into a new plate filled with 1 mL of extractant solution.
(vii) The plates were sealed using Parafilm® to stop evaporation and protected from light using foil before extract at 2-8° C. to minimise evaporation.
(viii) Following extraction the RHE tissue were removed from the wells and an additional 1 mL of extractant solution was added. Using a pipette the extraction solution was mixed and made visually homogenous.
(ix) The solution from Step (viii) was transferred in to n=3 wells (200 μL per well) using an automatic pipette of a 96 well plate and the optical density was measured at a wavelength of 570 nm.

3.2.6 Analysis of Cytokine Release

The conditioned media was analysed to determine the concentration of GM-CSF released. An Invitrogen Human GM-CSF kit (a solid phase sandwich Enzyme Linked-Immuno-Sorbent Assay (ELISA)) was utilised to quantify the concentration of GM-CSF. An antibody specific for human GM-CSF is coated onto the wells of a microtiter plate. Each sample, including standards of human GM-CSF, was pipetted directly into the coated wells, followed by the addition of a biotinylated second antibody. The plate was incubated under ambient conditions for 0.5 h during which period the human GM-CSF antigen binds simultaneously to the immobilised (capture) antibody on one site, and to the solution phase biotinylated antibody on a second site. The excess second antibody was removed and streptavidin-peroxidase (enzyme) was added. This enzyme binds to the biotinylated antibody to complete a four-member sandwich. The sandwich was incubated for a second time under ambient conditions for 0.5 h and any unbound enzyme was removed by washing, following which a substrate solution was added, which results in the formation of a coloured product that was quantified by measuring the absorbance of the solution at 450 nm using a μQuant spectrophotometer. The intensity of this coloured product is directly proportional to the concentration of human GM-CSF present in the original sample, and is quantified from the provided GM-CSF standards.

### 3.3 Results and Discussion

FIGS. 15 and 16 illustrate the effect of treatment with 10% w/v, N-H-S in water, 1% w/v, N-H-G in water, 10% w/v, L-h-S in water and a 10% w/v combination 1:1:1 (N-H-S:N-H-G: L-h-S) solution over 6 days in both psoriasis and control reconstructed human skin models. This demonstrates that release of GM-CSF from psoriasis tissues is reduced by treatment with N-H-S, L-h-S, N-H-G and combination solutions compared to untreated psoriasis tissues. This reduction in GM-CSF release suggested that N-hydroxyserine (N-H-S), L-homoserine (L-h-S), N-hydroxyglycine (N-H-G) and a 1:1:1 combination could be beneficial in the treatment of psoriasis.

None of the test solutions 10% w/v, N-H-S in water, 1% w/v, N-H-G in water, 10% w/v, L-h-S in water and a 10% w/v combination 1:1:1 (N-H-S:N-H-G:L-h-S) solution had a strong negative impact on tissue viability over 6 day experimental period. This data supports the conclusions from the irritation assessments that the solutions are non-irritating i.e. tissue viability remained above 50% for the duration of the experiment (Table 6).

TABLE 6
Tissues viability after exposure to test items (n = 1)

| | Normal RHE tissue viability | | | Psoriatic tissues as a | | |
|---|---|---|---|---|---|---|
| | as a percentage of untreated | | | percentage of untreated | | |
| | control (%) | | | control (%) | | |
| | Number of days in treatments | | | | | |
| | 2 | 4 | 6 | 2 | 4 | 6 |
| 10% w/v N-H-S | 83.4 | 91.5 | 86.9 | 94.0 | 75.3 | 96.0 |
| 10% w/v L-h-S | 88.3 | 82.1 | 78.6 | 85.9 | 88.8 | 81.2 |
| 10% w/v N-H-G | 97.9 | 91.5 | 93.3 | 87.1 | 77.6 | 89.6 |
| 10% w/v | 82.2 | 85.5 | 75.5 | 101.0 | 84.5 | 101.3 |
| Combination | | | | | | |
| CTRL | 100.0 | 100.0 | 100.0 | 100.0 | 100.0 | 100.0 |

FIG. 15 shows release of GM-CSF from psoriasis tissues. Each point represents the average cumulative release of GM-CSF over 6 day with measurements made at 2, 4, and 6 days with error bars representing the range n=2-3.

FIG. 16 shows total release of GM-CSF. Each bar represents the total release of GM-CSF over 6 day from both psoriasis model and control RHE tissues. Error bars representing the range n=2-3.

### 4 Summary

### 4.1 RHE Irritation Assessment (Method 1)

The skin irritation potential of L-homoserine (L-h-S) and alpha-hydroxyglycine (α-H-G) was investigated using the 42 bis assay protocol with the addition of an increased dosing period of 24 h. Analysis of the tissue viability after both incubation periods (42 min and 24 h) demonstrated the tissue viability remained over 50% which suggested that L-homoserine (L-h-S) and alpha-hydroxyglycine (α-H-G) are both non-irritants.

### 4.2 RHE Irritation Assessment (Method 2)

To further investigate the potential for skin irritation in response to N-hydroxyglycine (N-H-G) and N-hydroxyserine (N-H-S) the ET-50 skin irritation protocol (MatTek) was used. This was selected as it is suitable for formulations and mixtures. In this assay in addition to the test item N-hydroxyglycine (N-H-G) and N-hydroxyserine (N-H-S) two commercially available amino acid related compounds were selected for comparison (L-serine and glycine). In this study alpha-hydroxyglycine (α-H-G) was found to be a non-irritant (i.e. had an ET-50<24 h) and 10% w/v N-hydroxyserine (N-H-S) was categorised as a very mild irritant (i.e. had an ET-50 between 12-24 h). Both of the commercially available amino acid related compounds (L-serine and glycine) were shown to be non-irritants.

### 4.3 In Vitro Assessment of Psoriatic Tissue

The effect of N-hydroxyserine (N-H-S), L-homoserine (L-h-S), N-hydroxyglycine (N-H-G) on GM-CSF release in an RHE model for psoriasis was investigated. It was observed that N-hydroxyserine (N-H-S), L-homoserine (L-h-S), N-hydroxyglycine (N-H-G) and the combination treatment reduced the production of GM-CSF which suggests that N-hydroxyserine (N-H-S), L-homoserine (L-h-S), N-hydroxyglycine (N-H-G) may be beneficial in the treatment of psoriasis. Thus, there is provided the use of amino acids of the present invention as anti-inflammatory agents, especially where a contributory factor in said inflammation is GM-CSF. Particularly preferred amino acids for such use are N-hydroxyserine, L-homoserine, N-hydroxyglycine, and combinations containing one or more thereof.

The tissue viability of the RHE model for psoriasis during dosing with N-hydroxyserine (N-H-S), L-homoserine (L-h-S), N-hydroxyglycine (N-H-G) was in agreement with the previous studies for both L-homoserine (L-h-S), N-hydroxyglycine (N-H-G) further confirming them as non-irritants, as RHE viability remained above 50% of the untreated controls over a 6 day dosing period. This study also suggested that N-hydroxyserine (N-H-S) was a non-irritant.

Thus, there is provided the use of amino acids of the present invention as anti-irritants. Particularly preferred amino acids for such use are N-hydroxyserine, L-homoserine, N-hydroxyglycine, and combinations containing one or more thereof.

### REFERENCES

Clar E J, Fourtanier A (1981). Pyrrolidone carboxylic acid and the skin [in French]. Int J Cosmet Sci. 3: 101-13.
Harding C R, Watkinson A, Rawlings A V, et al (2000). Dry skin, moisturisation and corneodesmolysis. Int J Cosmet Set. 22: 21-52.
Horii I, Nakayama Y, Obata M, et al (1989). Stratum corneum hydration and amino acid content in xerotic skin. Brit J Dermatol. 121: 587-592.
Jokura Y, Ishikawa S, Tokuda H, Imokawa G (1995). Molecular analysis of elastic properties of the stratum corneum by solid state ¹³C-nuclear magnetic resonance spectroscopy. J Invest Dermatol 104: 806-812.
Kezic S, Kemperman P M, Koster E S, De Jongh C M, Thio H B (2008). Loss-of-function mutations in the filaggrin gene lead to reduced level of natural moisturizing factor in the stratum corneum. J Invest Dermatol 128: 2117-2119.
Kezic S, O'Regan G M, Yau N, et al (2011). Levels of filaggrin degradation products are influenced by both filaggrin genotype and atopic dermatitis severity. Allergy 66: 934-40.
Nakagawa N, Sakai S, Matsumoto M, Yamada K (2004). Relationship between NMF (lactate and potassium) content and the physical properties of the stratum corneum in healthy subjects. J Invest Dermatol 122: 755-763.
Palmer C N, Irvine atopic dermatitis, Terron-Kwiatkowski A, et al (2006). Common loss of-function variants of the epidermal barrier protein filaggrin are a major predisposing factor for atopic dermatitis. Nat Genet. 38: 441-446.
Rawlings A V, Scott I R, Harding C R, et al (1994). Stratum corneum moisturisation at the molecular level. J Invest Dermatol. 103: 731-741.
Sasaki O, Kanai I, Yazawa Y (2007). Relationship between the chemical structure of humic substances and their hygroscopic properties. Science 1: 17-22.
Scott I R, Harding C R, Barrett J G (1982). Histidine-rich protein of the keratohyalin granules. Source of the free amino acids, urocanic acid, and pyrrolidone carboxylic acid in the stratum corneum. Biochim Biophys Acta. 719: 110-117.
Weber, M. T., et al (2012). J Clin Aesthet Dermatol. 5: 29-39.
White-Chu E F, Reddy M. Dry skin in the elderly: complexities of a common problem. Clin Dermatol. 2011; 29(1):37-42.

1. An unnatural, hygroscopic amino acid for use in enhancing hydration and/or the moisture retention and/or uptake properties of an external keratinaceous structure of an animal.

2. An unnatural, hygroscopic amino acid for use in accordance with claim 1, wherein said structure is the skin.

3. An unnatural, hygroscopic amino acid for use according to claim 1 wherein the amino acid is capable of absorbing and retaining moisture from the atmosphere at a relative humidity (RH) of ≦50%, at 32° C.

4. An unnatural, hygroscopic amino acid for use according to claim 3, wherein the amino acid is capable of absorbing and retaining moisture from the atmosphere at a relative humidity (RH) of ≦40%, at 32° C.

5. An unnatural, hygroscopic amino acid for use according to claim 1, wherein said host is a mammal.

6. An unnatural, hygroscopic amino acid for use according to claim 5, wherein said host is a human.

7. A topical, including ocular, formulation comprising at least one amino acid as defined in claim 1.

8. A formulation according to claim 7, further comprising one or more additional moisturising ingredients.

9. A formulation according to claim 7, further comprising one or more additional further amino acids.

10. A formulation according to claim 7, wherein said formulation mimics NMF, by using one or more amino acid ingredients of NMF.

11. A formulation according to claim 10, wherein said one or more amino acid ingredients of NMF are present in amounts and/or ratios approximating those found in NMF.

12. A formulation according to claim 7, wherein said formulation mimics NMF, by using one or more non-amino acid ingredients of NMF.

13. A formulation according to claim 12, wherein said one or more non-amino acid ingredients of NMF are present in amounts and/or ratios approximating those found in NMF.

14. A formulation according to claim 12, wherein said one or more non-amino acid ingredients of NMF include one or more salts.

15. A formulation according to claim 14, wherein said one or more salts are selected from the sodium, potassium, HCl, and diethylamine salts.

16. An amino acid for use according to claim 1, or a composition according to claim 7, wherein the amino acid is selected from N-hydroxyserine, N-hydroxyglycine, L-homoserine, α-hydroxyglycine, 2-(aminooxy)-2-hydroxyacetic acid, 2-hydroxy-2-(hydroxyamino) acetic acid, 2-(aminooxy)acetic acid, and combinations thereof.

17. An amino acid according to claim 16, which is selected from α-hydroxyglycine, or L-homoserine, or both.

18. An amino acid, or a composition comprising said amino acid, as defined in claim 1, for use in the cosmetic treatment of an external keratinaceous structure of an animal.

19. An amino acid according to claim 18, wherein said structure is the skin.

20. An amino acid according to claim 18, for a use selected from the group consisting of:
a) use in emollient formulations;
b) use in ocular formulation for the treatment of dry eye;
c) use to moisturise thickened skin;
d) use in nail and/or hoof softening;
e) use in conjunction with hair removal techniques;
f) use in face packs;
g) use to hydrate the skin in the treatment of skin conditions; and
h) and use in hydrating the skin before application of a barrier preparation.
i) use in wound healing

21. An amino acid as defined in claim 1, for use in facilitating transdermal absorption of drugs.

22. An amino acid, or a composition comprising said amino acid, as defined in claim 1, for use as an anti-inflammatory agent, optionally in combination with a further anti-inflammatory agent.

23. An amino acid, or a composition, according to claim 22, wherein a contributory factor in said inflammation is GM-CSF.

24. An amino acid, or a composition, according to claim 22, wherein said amino acid is selected from N-hydroxyserine, L-homoserine, N-hydroxyglycine, and combinations containing one or more thereof.

25. An amino acid, or a composition comprising said amino acid, as defined in claim 1, for use as an anti-irritant.

26. An amino acid, or a composition, according to claim 25, wherein said amino acid is selected from N-hydroxyserine, L-homoserine, N-hydroxyglycine, and combinations containing one or more thereof.

27. A composition for transdermal administration of a drug, comprising an amino acid as defined in claim 1, to assist in dermal penetration of said drug

28. A composition for transdermal administration of a drug, comprising an amino acid as defined in claim 1, to reduce the irritation of said drug.

29. An amino acid, or a composition comprising said amino acid, as defined in claim 1, wherein said amino acid has a deliquescence relative humidity (DRH) of no greater than 80% at 32° C.

30. An amino acid, or a composition comprising said amino acid, as defined in claim 1, wherein said amino acid has an O/C ratio of at least 0.7.
